(19) United States
(12) Patent Application Publication
(10) Pub. No.: US 2023/0310663 A1
(43) Pub. Date: Oct. 5, 2023

(54) METHOD OF SYNTHESIZING 18F RADIOLABELED BIOMOLECULAR AGENTS

(71) Applicants: ARIZONA BOARD OF REGENTS ON BEHALF OF THE UNIVERSITY OF ARIZONA, Tucson, AZ (US); Theracea Pharma, LC, Tucson, AZ (US)

(72) Inventors: Iman Daryaei, Tucson, AZ (US); Houng Dang, Tucson, AZ (US); Nathan Wright, Tucson, AZ (US); Julian Dresler, Tucson, AZ (US)

(21) Appl. No.: 18/301,896

(22) Filed: Apr. 17, 2023

Related U.S. Application Data
(63) Continuation-in-part of application No. 16/493,668, filed on Sep. 12, 2019, now Pat. No. 11,628,229.
(60) Provisional application No. 62/470,735, filed on Mar. 13, 2017.

(51) Int. Cl. A61K 51/04; A61K 49/08; C07B 59/00

(52) U.S. Cl. CPC A61K 51/0497; A61K 49/085; C07B 59/00; C07B 2200/05

(57) ABSTRACT

Methods of preparing ¹⁸F targeting biomolecules and small molecules with biological activity for therapeutic and/or diagnostic applications using fluorinated aromatic compounds. A fluorinated conjugated target tracer is synthesized and purified with temperature and solvent conditions that are mild for the tracer molecule. The purified fluorinated-conjugated target tracer is then labeled with ¹⁸F using ¹⁸F salts within a short reaction time, and with temperature and solvent conditions that are mild for the tracer molecule. The method provides a quick and convenient process that maintains the biological activities of the target molecules. The radio-labeled biomolecules may be used as contrast agents for Positron Emission Tomography (PET).

### CROSS REFERENCE

This application is a continuation-in-part and claims benefit of U.S. patent application Ser. No. 16/493,668 filed Sep. 12, 2019, which is a 371 of PCT/US2018/022160 filed Mar. 13, 2018, which claims benefit of U.S. Provisional Application No. 62/470,735, filed Mar. 13, 2017, the specifications of which are incorporated herein in their entirety by reference.

### FIELD OF THE INVENTION

The present invention features compositions and methods of preparing ¹⁸F targeting biomolecules and small molecules with biological activity for therapeutic and/or diagnostic applications using fluorinated aromatic compounds. The methods described herein can conveniently and rapidly label biomolecules and other small molecules with 18-fluorine (¹⁸F), a radioactive version of the fluorine atom.

### BACKGROUND OF THE INVENTION

Positron emission tomography (PET) is a type of nuclear medicine imaging that utilizes small amounts of radioactive material, called radiopharmaceuticals or radiotracers, to diagnose and evaluate medical conditions, including cancers, heart disease, neurological disorders, and other abnormalities within the body. In particular, the PET procedure can evaluate the metabolism of a specific organ or area of the body, and provide information about its physiology, anatomy, and biochemical properties. The radiotracer may be injected, swallowed, or inhaled into the body, and it accumulates in the organ or area of the body being examined. As the radiotracer decays, an imaging device detects its radioactive emissions, namely, positron emissions, and produces an image map from which said information can be evaluated.

Conventional radiotracers may comprise a molecule radiolabeled with a radioactive atom, such as ¹⁵O, ¹⁸F, ¹¹C, or ¹³N. Of said radioactive atoms, fluorine-18 (¹⁸F) is most preferred due to its longer half life (<120 min), as compared to the half lives of the other atoms; for instance, 11C has a half life of about 20 minutes. The longer half life of ¹⁸F allows for chemical reactions with ¹⁸F and other compounds to produce the radiotracer, and further allows for longer PET examinations. There is a growing interest in the radiolabeling of biomolecules, such as antibodies, minibodies, scFv, mRNA, siRNA, DNA, carbohydrates, peptides, glycoproteins, and the like, with a radionuclide, such as ¹⁸F, in order to produce a highly-specific targeting PET tracer. For example, a radioactive atom may be applied to glucose to produce a radiotracer for a PET scan of the brain.

Producing ¹⁸F requires the use of a cyclotron. The ¹⁸F ion must then be chemically incorporated into a molecule, purified, and administered to the subject. In addition, since the radiotracer will require some time to accumulate at the target organ or area, this process must be performed rapidly and efficiently such that there is a sufficient amount of the ¹⁸F radioisotope still active for producing a quality image of the target organ or area. Due to the relatively harsh reaction conditions, such as high temperatures and harsh solvents, to incorporate the ¹⁸F ion, direct radiofluorination is usually incompatible with the biomolecule, and would further require an intermediate compound for radiolabeling the biomolecule. Again, this method must be done in a relatively short time frame to ensure that there is sufficient radioactivity in the ¹⁸F radioisotope for imaging purposes.

In some aspects, ¹⁸F-labeling of biomolecules can be performed in two steps. First, ¹⁸F-labeling of a prosthetic group is carried out, followed by a conjugation reaction with the target biomolecule in the second step. ¹⁸F-labeling of prosthetic groups are preferably performed in a polar aprotic solvent (ACN, DMSO, or DMF) or in some instances polar protic solvents such at MeOH, EtOH, n-PrOH, n-BuOH, i-BuOH, or i-PrOH with or without presence of water (up to 15%). Solvents are used to dissolve solutes (reactants and ¹⁸F-ions) for a more effective labeling process. Since water has very strong interaction with fluoride ions, water solvated fluoride is not a strong nucleophile for the labeling reactions. Therefore, an organic solvent or a combination of organic solvents is also used to disturb water-fluoride interaction to allow for a better nucleophilic reaction. Considering the limited solubility of ¹⁸F-ions in organic solvents, high temperature and addition of phase transfer catalysts (PTC) (e.g., K_{2.2.2}, TBAHCO₃, TMAHCO₃, etc) are used to effectively bring the ¹⁸F-ions into the organic phase. In addition, inorganic bases (such as CsCO₃, K₂CO₃, KHCO₃) are used to facilitate the labeling reaction. Depending on the nature of the prosthetic group and the mechanism for the reaction, use of high temperature provides the required energy to pass the activation energy barrier in the formation of the intermediates and finally the desired products.

One of the significant challenges in the ¹⁸F-labeling process is the relatively low concentration of ¹⁸F-ions after they are produced in a cyclotron. To overcome this challenge, the prosthetic groups with functional groups such as N₂, NMe₃, NO₂, I-Ph, Cl, Br, SnMe₃ are employed where the functional groups are suitable leaving groups that are replaced by ¹⁸F⁻. The replacement of the leaving group with ¹⁸F results in formation of prosthetic groups with different chemical properties compared to the reactants. Purification of the ¹⁸F-labeled prosthetic groups from the reactant yields in products with relatively high specific (or molar) activity. There are several pitfalls to using this approach for ¹⁸F-labeling of sensitive biomolecules: 1) the purification step usually requires advanced purification systems (e.g., high-performance liquid chromatography systems), 2) the purification process is time consuming and can result in loss of activity, 3) post-purification of the ¹⁸F-labeled prosthetic group is required (e.g., reducing the volume of solvent) to have the prosthetic group ready for conjugation, and 4) presence of inorganic strong bases in the labeling reactions requires extra caution when purification systems are used.

The presence of a chemical handle on the prosthetic group for bioconjugation to the biomolecule is another consideration when prosthetic groups for ¹⁸F-labeling of biomolecules are selected. The presence of a secondary functional group (such as aldehyde, ester, or a linker to aldehyde, ester, maleimide, azide, tetrazine, alkyne, etc.) requires precise design of the labeling condition (including amount of phase-transfer catalyst, base, temperature, and choice of solvent(s)) where ¹⁸F labeling occurs while the handle remains unmodified.

[¹⁸F]/¹⁹F exchange through a nucleophilic aromatic substitution (SNAr) provides an alternative approach for preparation of ¹⁸F-labeled small molecules, where there is no need for comprehensive purification of the reaction mixture to isolate the ¹⁸F-labeled product from the starting materials. Early studies by Chakraborty and Kilbourn, Tewson et al., Babich et al., Ludwig et al., and Langer et al. using this concept under harsh conditions (e.g., high temperature reactions in DMSO) allowed preparation of ¹⁸F-labeled small molecules. (Chakraborty, P. K., Kilbourn, M. R. [¹⁸F] Fluorination/Decarbonylation: New Route to Aryl [18F] Fluorides. Int. J. Raiat. Isot. 1991, 42(12), 1209-1213; Tewson, T. J., Yang, D., Wong, G., Macy, D., DeJesus, O. J., Nickels, R. J., Perlman, S. B., Taylor, M., Frank, P. The Synthesis of Fluorine-18 Lomefloxacin and Its Preliminary Use in Human Studies. Nuc. Med. Biol. 1996, 23, 767-772; Babich, J. W., Rubin, R. H., Graham, W. A., Wilkinson, R. A., Vincent, J., Fischman, A. J. 18F-Labeling and Biodistribution of the Novel Fluoro-Quinolone Antimicrobial Agent, Trovafloxacin (CP 99,219). Nuc. Med. Biol. 1996, 23, 995-998; Ludwig, T., Ermert, J., Coenen, H. H. 4-[18F] Fluoroarylalkylethers via an Improved Synthesis of n.c.a 4-[18F] Fluorophenol. Nuc. Med. Biol. 2002, 29, 255-262; Langer, O., Mitterhauser, M., Brunner, M., Zeitlinger, M., Wadsak, W., Mayer, B. X., Kletter, K., Muller, M. Synthesis of Fluorine-18-labeled Ciprofloxacin for PET Studies in Humans. Nuc. Med. Biol. 2003, 30, 285-291).

[¹⁸F]/¹⁹F exchange of perfluoroaryl (PFAr) compounds provides an easy and fast process for preparation of ¹⁸F-labeled prosthetic groups that can be easily conjugated to the biomolecules. There are two reports in the literature on use of PFAr for ¹⁸F-labeling purposes. Specifically, Blom performed ¹⁸F-labeling optimization on a derivatives of PFAr compounds in different concentrations using K_{2.2.2}/K₂CO₃ system in DMSO at different temperatures (block heating and microwave heating) (Blom, E., Karimi, F., Langstrom, B. [¹⁸F]/¹⁹F exchange in fluorine containing compounds for potential use in ¹⁸F-labeling strategies J. Label Compd. Radiopharm 2009, 52 504-511). Then they applied the optimized condition on two small molecules that were prone to an ¹⁸F/¹⁹F exchange reaction (FIG. 19). However, the reported specific (molar) activity of the target small molecules seems very low. There is no further work by this group to use the [¹⁸F]/¹⁹F exchange for preparation of prosthetic groups or heat/solvent sensitive biomolecules.

In a similar manner, Jacobson showed ¹⁸F/¹⁹F exchange on hexafluorobenzene (HFB) in DMSO at room temperature followed by an isolation process (distillation) and consequently bioconjugation of the ¹⁸F-labeled HFB to a small peptide (Jacobson, O., Yan, X., Ma, Y., et al. Novel Method for Radiolabeling and Dimerizing Thiolated Peptides Using ¹⁸F-Hexafluorobenzene. Bioconjugate Chem. 2015, 26, 2016-2020). Jacobson et al. teaches a first approach in which a hexafluorobenzene molecule was conjugated to tetrafluoro-(1,4-phenylene)bis((4-chlorobenzyl)sulfane compound as a model reaction for conjugation of PFAr to small molecules (FIG. 20A). Then the ¹⁸F/¹⁹F exchange step was performed at a required reaction temperature of 90° C. to prepare the ¹⁸F-labeled derivative where the reaction time was 15 min and the yield was 33% (FIG. 20A). Although this method may work for ¹⁸F-labeling of heat-stable small molecules, Jacobson et al. deemed the ¹⁸F/¹⁹F exchange reaction an unsuitable method for general ¹⁸F-labeling of peptides due to the harsh reaction conditions (e.g., elevated temperatures) for biomolecules. In the end, Jacobson et al. further teaches a second approach in which the order of these two steps was switched (FIG. 20B). First, the ¹⁸F/¹⁹F exchange step was performed on a hexafluorobenezene compound according to the method that was introduced by Blom et al. and was adopted for automation in a modular system (Eckert and Ziegler). Then, the hot PFAr compound was purified by distillation with argon flow at 25° C. into a vial containing DMF cooled in dry ice/acetonitrile (−45° C.). Upon purification, the hot PFAr compound was reacted with thiolated c(RGDfK) peptide in presence of excess amount of TRIS base and TCEP reducing agent, which needed 20-25 minutes for 50% conjugation and then additional time for purification of the dimerized thiolated c(RGDfK) peptide (hot compound) (FIG. 20B). Two steps of purification (a purification step to isolate ¹⁸F—HFB and then the second purification step to isolate the final product) leads to the loss of activity. Considering that ¹⁸F/¹⁹F exchange by nature has low specific activity (due to the fact that 18F-labeled material has the same chemical properties as the unlabeled material and they can not be separated from each other), the need for two steps of purification limits the application of this technology.

Considering that there are multiple parameters that are involved in ¹⁸F-labeling of prosthetic groups (specifically PFAr), the present invention provides an efficient ¹⁸F-labeling process for preparation of ¹⁸F—PFAr as 1) a versatile prosthetic group for labeling of biomolecules and 2) a site on biomolecules for ¹⁸F labeling. This method uses radioactive ¹⁸F⁻ and does not require sophisticated radiochemistry, thereby can be readily adopted for preparation of a wide range of ¹⁸F-radiotracers based on biomolecules.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

The present invention features a methodology for the design and synthesis of MRI/¹⁸F-PET and/or ¹⁸F labeling of biomolecules. Embodiments of the invention are given in the dependent claims. Embodiments of the present invention can be freely combined with each other if they are not mutually exclusive.

In some aspects, the present invention features a process that can be adopted by different types of facilities for both manual and automated preparation of ¹⁸F-labeled radiotracers. The ¹⁸F-labeling approach can be applied on perfluoroaryl (PFAr) compounds pre- or post-bioconjugation for the preparation of ¹⁸F-labeled biomolecules.

According to some embodiments, the present invention features a method of preparing an ¹⁸F-labeled radiotracer for use in positron emission tomography (PET). The method may comprise providing an ¹⁸F compound, providing a target tracer compound having a biological moiety, and reacting the ¹⁸F compound with the target tracer compound in a solvent and at a temperature that is mild for the biological moiety, thereby forming the ¹⁸F-labeled radiotracer and preserving the biological activity of the biological moiety.

In one embodiment, the step of providing a target tracer compound having a biological moiety may comprise conjugation, and the step of reacting the ¹⁸F compound with the target tracer compound may comprise radiolabeling. In some embodiments, the step of providing the target tracer compound having the biological moiety may comprise reacting the target tracer compound with a non-radioactive fluorinated compound in a non-aqueous solvent or an aqueous solvent that is predominantly water, at a temperature that is mild for the biological moiety, thereby forming a fluorinated target tracer compound. The fluorinated target tracer compound is non-radioactive, and a biological activity of the biological moiety is preserved.

In some embodiments, the ¹⁸F compound is an ¹⁸F salt. Non-limiting examples of the ¹⁸F salt include K¹⁸F, Na¹⁸F, Cs¹⁸F, ¹⁸F—K_{2.2.2}/K₂CO₃, or a crown ether, ¹⁸F—NR₄ (where R is a methyl, ethyl, propyl, butyl, or pentyl). In some embodiments, the temperature is at most 60° C. In some embodiments, the solvent in which the ¹⁸F compound is reacted with the target tracer compound comprises a second solvent that is a non-aqueous solvent or an aqueous solvent that is predominantly water. In some embodiments, the first aqueous solvent, the second aqueous solvent, or both may further comprise a co-solvent. In some embodiments, the co-solvent is DMSO, DMF, ACN, EtOH, MeOH, iPrOH, PrOH, t-BuOH, THF, DEE, DCM, acetone, or a combination thereof.

According to another embodiment, the step of providing the ¹⁸F compound may comprise radiolabeling, and the step of reacting the ¹⁸F compound with the target tracer compound may comprise conjugation. In some embodiments, the step of providing the ¹⁸F compound may comprise reacting an ¹⁸F salt with a non-radioactive fluorinated compound such that said fluorinated compound is ¹⁸F-labeled, thereby forming the ¹⁸F-compound. In other embodiments, the step of providing the ¹⁸F compound may further comprise prior to reacting the ¹⁸F salt with the non-radioactive fluorinated compound, conjugating the non-radioactive fluorinated compound to a linker with an active functional group.

In some embodiments, the non-radioactive fluorinated compound has a functional group that acts as a linker for direct or indirect conjugation to the target tracer compound. In some embodiments, the target tracer compound has a functional group that reacts with the ¹⁸F-labeled compound via aromatic nucleophilic substitution. In other embodiments, the ¹⁸F salt is K¹⁸F, Na¹⁸F, Cs¹⁸F, ¹⁸F—K_{2.2.2}/K₂CO₃, a crown ether, or ¹⁸F—NR₄ (where R is a methyl, ethyl, propyl, butyl, or pentyl).

In accordance with the embodiments described herein, the fluorinated compound may be any one of the following formulas:

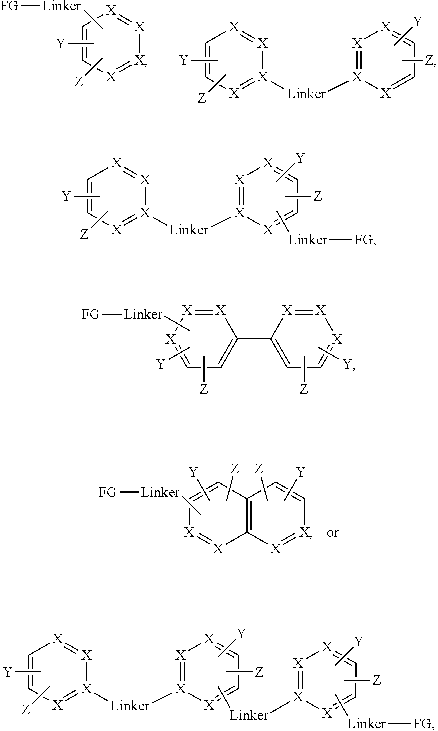

In some embodiments, X is C or N, Y is F, and Z is Cl, Br, I, NO₂, N₂, Ns, CO—NH₂, SH, SO₃H, COOH, COOR, or NR₃, where R is a methyl, ethyl, propyl, butyl, pentyl, or their isomers, and where FG is maleimide, NHS-ester, azide, tetrazine, alkyne, or alkene.

In some embodiments, the Linker is optional or is SO₂—, SO—, CO—, CO—NH—, —O—, —S—, COO, —(CH₂CH₂O)ₙ—, —(CO-A-NH)ₙ—, —(CO—CH(CHCH₂OH)—NH)ₙ—, —(CO—CH₂—NH)ₙ—, —(CO—CH(CH₃)—NH)ₙ—, —COC₆H₄—, —CH₂CONH—, —NCCCC₆H₄—, —NHCO—, or —NHCS—, where A is —(CH₂)ₙ—, and n ranges from 1 to 10.

In some embodiments, the target tracer compound may comprise scFv, minibody, diabody, nanobody, and affibody, hormones, antibodies, glycoproteins, peptides, mRNA, siRNA, snRNA, DNA, or fragments thereof, carbohydrates, polycarbohydrates, cofactors, coenzymes, phospholipids, glycoproteins, hormones, polyethylene glycols (PEG), PEGylated biologics, PEGylated phospholipids, magnetic resonance imaging (MRI) agents, ultrasound agents, x-ray agents, computerized tomography (CT) agents, fluorescent agents, or synthetic organic or inorganic small molecules.

In a non-limiting embodiment, the method may comprise synthesizing and purifying a PFAr-conjugated target tracer, where the tracer includes a molecule, such as a biomolecule, that can only undergo reactions under mild conditions. The purified PFAr-conjugated target tracer is then labeled with ¹⁸F using ¹⁸F salts within a short reaction time. Excess ¹⁸F salts can be removed using a simple dialysis or chromatography using SEP-PAK columns. Preferably, the conjugation reactions may be performed by a skilled chemist, whereas the ¹⁸F radiolabeling may be done by experts and even non-experts in a radiopharmacy or where a cyclotron is located.

According to other embodiments, the present invention provides a kit for preparing an ¹⁸F-labeled radiotracer for use in positron emission tomography (PET). The kit may comprise an ¹⁸F compound, a target tracer compound having a biological moiety, and a set of instructions for preparing the ¹⁸F-labeled radiotracer prior to use in PET such that the biological activity of the biological moiety is preserved. In some embodiments, the set of instructions may comprise an instruction for reacting the ¹⁸F compound with the target tracer compound in a solvent and at a temperature that is mild for the biological moiety, thereby forming the ¹⁸F-labeled radiotracer, and preserving the biological activity of the biological moiety.

According to some other embodiments, the present invention provides a fluorinated precursor for use in preparing an ¹⁸F-labeled radiotracer. In one embodiment, the fluorinated precursor may comprise an antibody fragment conjugated to a fluorinated compound via reaction on an aromatic ring of the fluorinated compound through a side chain of an amino acid or unnatural amino acid of the antibody fragment. In other embodiments, the fluorinated precursor may be any of the compounds disclosed herein, including those shown in FIGS. 5 and 7-13.

One of the unique and inventive technical features of the present invention is the method involves relatively mild reaction conditions that are tolerable by biomolecules. In particular, the PFAr moiety that is attached to the biomolecule or other target tracer undergoes a ¹⁹F-to-¹⁸F substitution under mild temperatures and solvent conditions that do not harm the biological moiety. Without wishing to limit the invention to any theory or mechanism, it is believed that the technical feature of the present invention advantageously provides for a quick procedure to radiolabel the biomolecules while retaining their biological activities. None of the presently known prior references or work has the unique inventive technical feature of the present invention.

Another unique and inventive technical feature of the present invention is the use of water-soluble PFAr compounds. Alternatively, the present invention features methods of modifying PFAr compounds to increase their water-solubility. Without wishing to limit the invention to any theory or mechanism, it is believed that the technical feature of the present invention advantageously allows for relatively mild reaction conditions that maintains the biological activities of the biomolecules. Again, none of the presently known prior references or work has the unique inventive technical feature of the present invention.

### BRIEF DESCRIPTION OF THE DRAWINGS

The features and advantages of the present invention will become apparent from a consideration of the following detailed description presented in connection with the accompanying drawings in which:

FIG. 1 shows a non-limiting embodiment of a synthesis process of the present invention.

FIG. 2 shows another non-limiting embodiment of a synthesis process.

FIG. 3 shows non-limiting examples of perfluoroaryl (PFAr) compounds that may be used in accordance with the present invention.

FIGS. 4A-4C show ¹⁸F-labeling of pentafluoropyridine analysis with an HPLC system, a 254 nm and a 280 nm UV detector, and a radio-counter and UV detector.

FIG. 5 is a non-limiting reaction of post conjugation labeling of duramycin (¹⁸F-duramycin) as a new radiotracer for the detection of phosphatidylethanolamine (PE).

FIG. 6 shows characterization and confirmation of the ¹⁸F-duramycin compound with HPLC system equipped with a UV detector and a radio-counter detector.

FIG. 7 shows non-limiting examples of precursor radiotracers for the detection of HER2 biomarkers.

FIG. 8 shows non-limiting examples of precursor radiotracers of PFAr-conjugated octreotide for the detection of somatostatin receptors.

FIG. 9 shows non-limiting examples of precursor radiotracers for the detection of granzyme B enzyme.

FIG. 10 shows non-limiting examples of precursor radiotracers for the detection of PSMA biomarkers.

FIG. 11 shows non-limiting examples of precursor radiotracers for therapeutic and diagnostic (do-called theranostic) applications based on HER2 biomarkers.

FIGS. 12A-12B show non-limiting examples of the preparation of precursor radiotracers with PFAr and ¹⁸F-radiolabelling of PFAr-precursors.

FIG. 13 is a non-limiting example of PFAr-linker with functional groups to conjugate to biomolecules.

FIGS. 14A-14B show characterization and confirmation of the PFAr-linker with a maleimide group for conjugation to cysteine amino acids by ¹⁹F-NMR and mass spectroscopy.

FIG. 15 a non-limiting example of PFAr-linker conjugated to a biomolecules (e.g. antibody fragment).

FIG. 16 shows characterization and confirmation of PFAr-linker conjugated to a biomolecule by mass spectroscopy.

FIGS. 17A-17C show characterization and confirmation of the PFAr-linker with a maleimide group for conjugation to cysteine amino acids with HPLC system equipped with a UV detector and a radio-counter detector.

FIGS. 18A-18B demonstrate uptake of a ¹⁸F—PFAr-HER2 biomolecules by Sk-Br-3 and MDA-MB-231 (control) cell lines.

FIG. 19 shows prior compounds disclosed in Blom et al., where compounds A and B were labeled with ¹⁸F in DMF or DMSO at 150° C. for 15 min. Compound C did not get labeled with ¹⁸F under the same conditions.

FIG. 20A shows prior reactions from Jacobson et al., where hexafluorobenzene is conjugated to a small molecule and then a ¹⁸F/¹⁹F exchange is performed in DMSO at 90° C.

FIG. 20B shows an alternative ¹⁸F/¹⁹F exchange method for ¹⁸F-labeling of biomolecules from Jacobson et al., where labeling was performed first and then conjugation to a peptide followed.

FIGS. 21A-21D show the conjugation of ¹⁸F-pentafluoropyridine to antibody fragments. FIG. 21A shows a representative reaction for labeling of biomolecules with a PFAr prosthetic group, and FIGS. 21B-21D show the confirmation and characterization of the labeled products by HPLC equipped with a radio-counter.

### DESCRIPTION OF PREFERRED EMBODIMENTS

As used herein, the term “labeling” or “radiolabeling”, and their derivatives refers to when an ¹⁸F radioisotope replaces any of the ¹⁹F isotopes in a fluorinated compound.

As used herein, a “target tracer compound”, or alternatively, “a target tracer molecule”, or simply, a “target tracer”, refers to a species that includes a moiety, such as a biomolecule, that can only be used in reactions under mild conditions, and is radiolabeled for use as a radiotracer (i.e. radiolabeled target tracer) in an imaging procedure, such as PET. The target tracer is distinct from and should not be confused nor interchanged with an intermediate compound, which refers to a prosthetic or carrier molecule that may be used to incorporate the radioactive atom into the target tracer, but has no direct use as a radiotracer in the imaging procedure.

As used herein, “biomolecules” refers to biologics, organic, or inorganic molecules, including small and large molecules, with biological activity. Non-limiting examples of biomolecules include protein/antibody fragments such as scFv, minibody, diabody, nanobody, and affibody, hormones, antibodies, glycoproteins, peptides, mRNA, siRNA, snRNA, DNA and fragments thereof, carbohydrates, polycarbohydrates, cofactors, coenzymes, phospholipids, glycoproteins, hormones, polyethylene glycols (PEG), PEGylated biologics, PEGylated phospholipids, magnetic resonance imaging (MRI) agents, ultrasound agents, x-ray agents, computerized tomography (CT) agents, fluorescent agents, and synthetic organic or inorganic small molecules.

As used herein, the term “mild” refers to reaction conditions in which the biological activity of the moiety in the target tracer compound is maintained and unaffected by said conditions. For example, a mild reaction condition can be achieved by refraining from use of radiation, heat, and harsh compounds such as strong acids, strong bases, concentrated inorganic salts, and/or volatile solvents. Without wishing to limit the present invention to a particular theory or mechanism, using mild reaction conditions can prevent denaturation of a protein molecule, thereby maintaining its native conformation.

In some embodiments, a mild temperature refers to a temperature in the range of about 15-40° C. or ambient temperature. In other embodiments, a mild temperature refers to a temperature that is about 60° C. or less. In some embodiments, a mild temperature refers to a temperature in the range of about 15-25° C., or about 20-40° C., or about 35-50° C., or about 45-60° C.

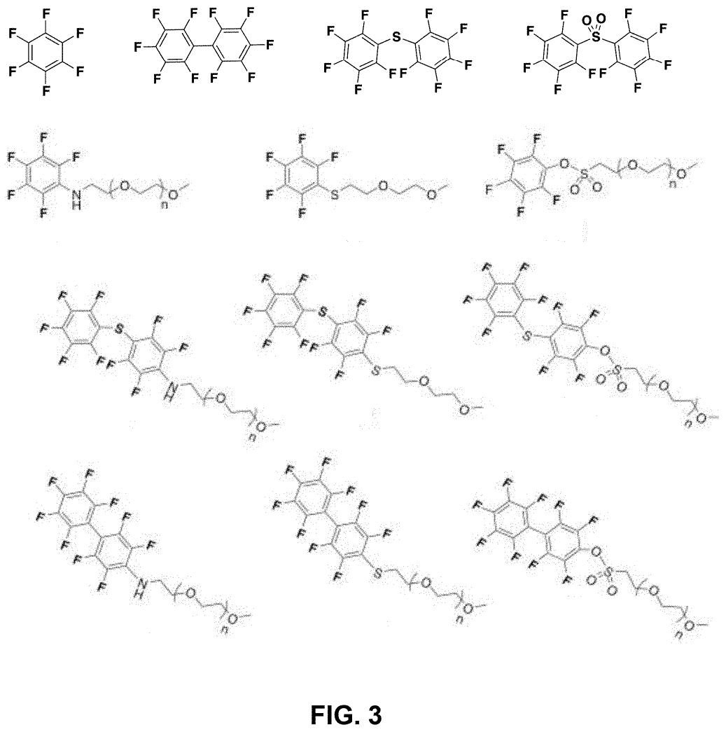

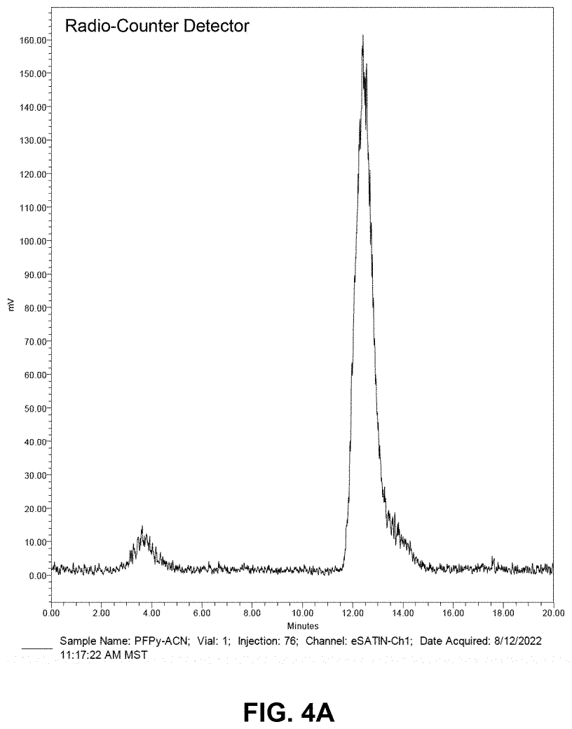

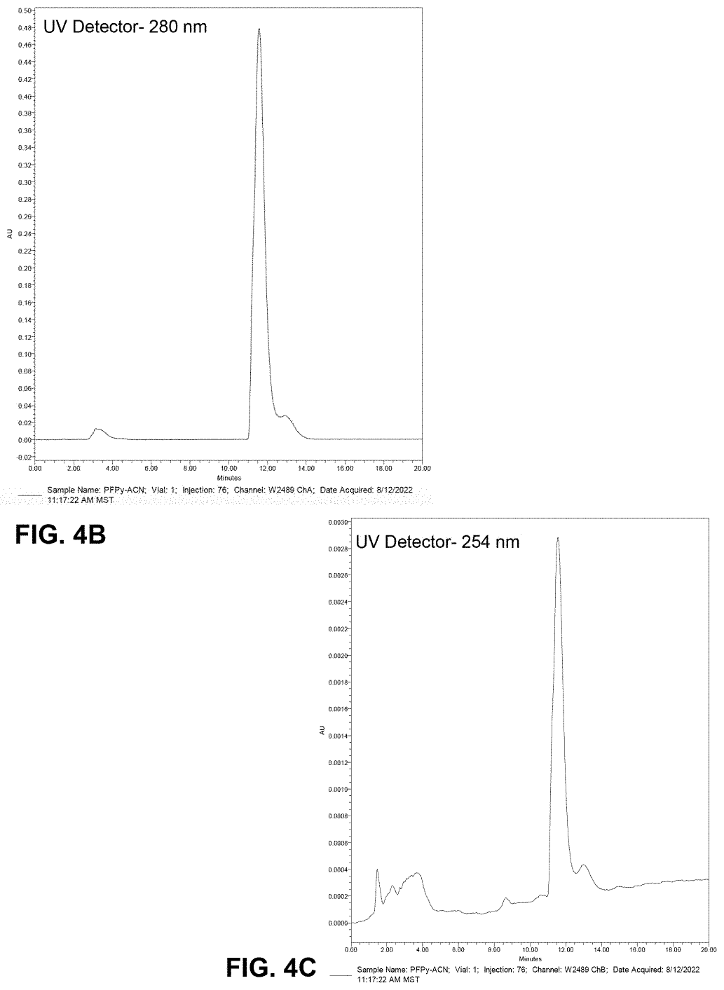

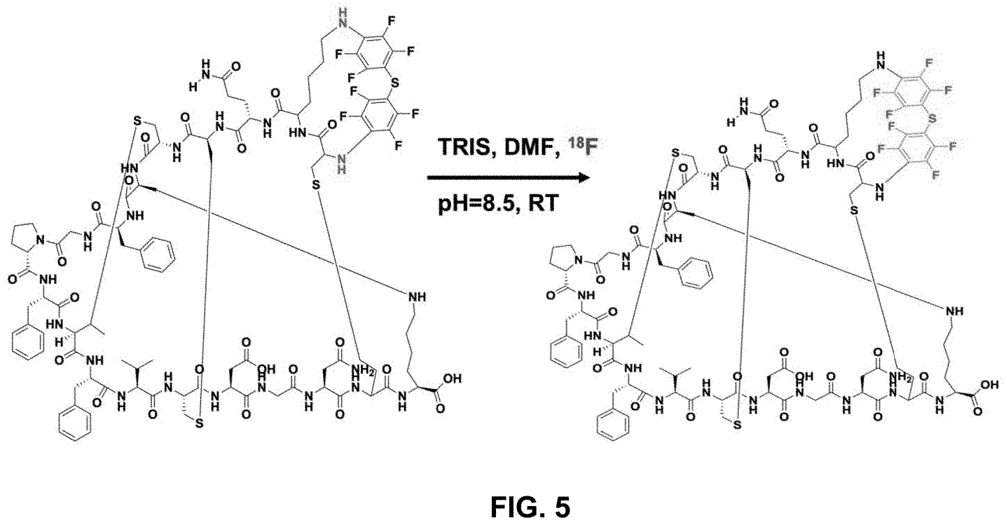

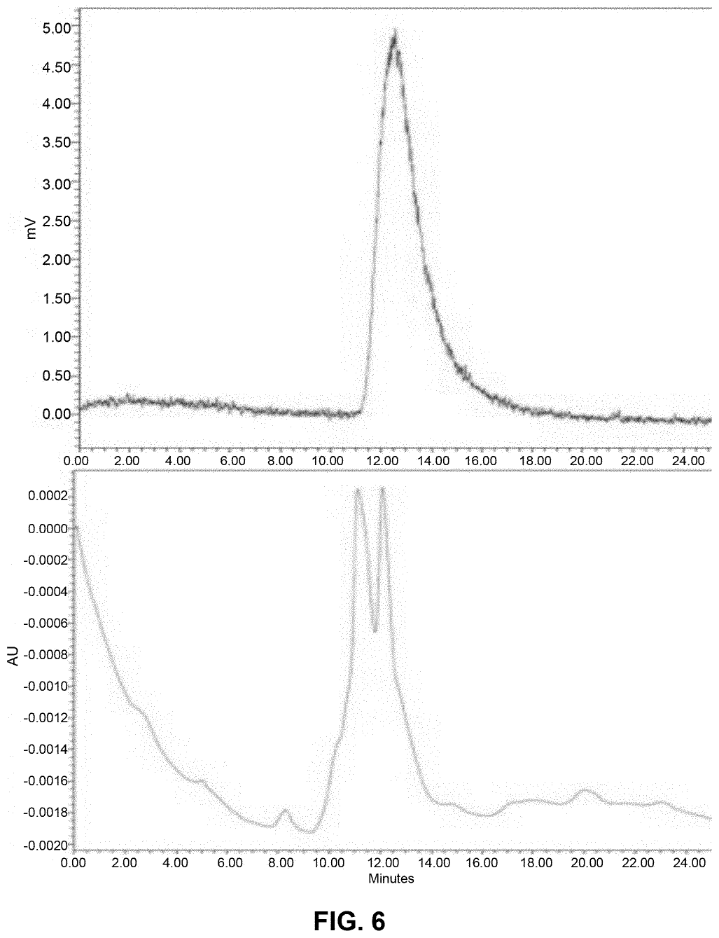

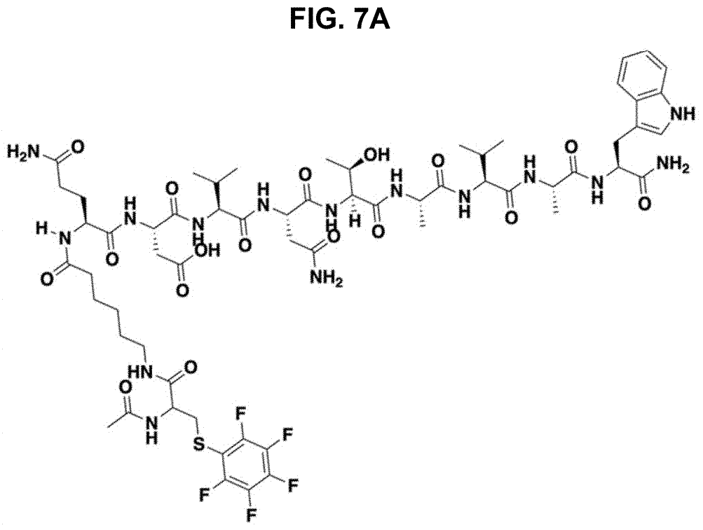

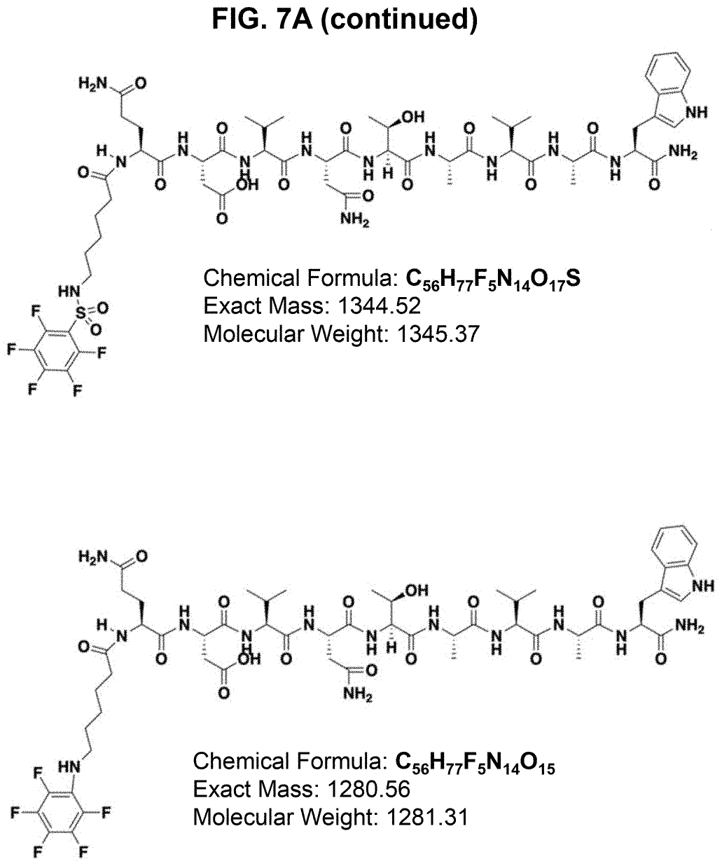

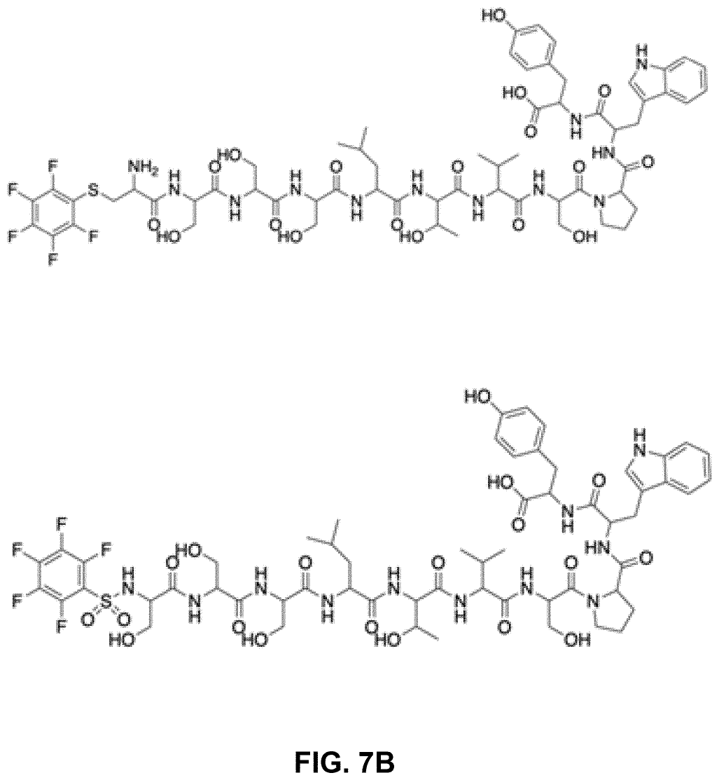

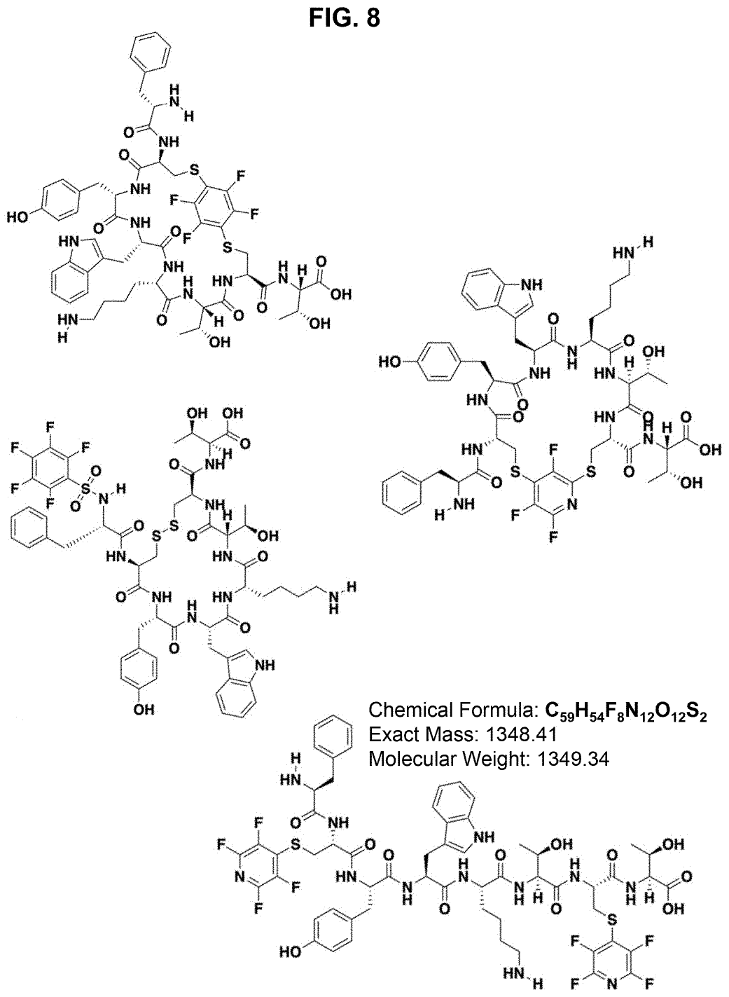

As used herein, a perflouroaryl (PFAr) compound refers to a fluorinated molecule comprising a plurality of fluorine atoms attached to an aromatic ring or aromatic ring system. The number of fluorine atoms may range from 2-6 per ring. In some embodiments, a variety of PFAr compounds may be used in accordance with the present invention. Examples of the PFAr compounds include, but are not limited to, the compounds shown in FIG. 3. In some embodiments, ‘n’ can range from 2-8.

In some embodiments, the solubility and reaction rate of the PFAs can vary for each compound. Without wishing to limit the invention to a particular theory or mechanism, the PFAr compounds may be modified to alter their solubility and reaction rate. For example, a PFAr compound having a poor solubility may be connected to water-soluble linkers, such as amino or thiol PEG, thereby increasing its solubility in aqueous solutions. Hence, it is another objective of the present invention to provide for water soluble PFAr compounds for use in the methods described herein.

In some embodiments, the present invention aims to provide methods of synthesizing of MRI/¹⁸F-PET and/or ¹⁸F labeling of biomolecules for ¹⁸F-PET imaging. The methods that will be described herein feature reactions under mild conditions that are tolerable by biomolecules, such as antibodies, minibodies, scFv, mRNA, siRNA, DNA, carbohydrates, and glycoproteins.

Referring now to the figures, in some embodiments, the present invention features a biomolecule conjugated to a fluorinated aromatic compound, and labeled with ¹⁸F in a mild reaction condition where biological properties of the biomolecule remain intact. In some embodiments, the reaction occurs in the presence of a solvent(s), ¹⁸F salt, other additives, and/or in a temperature between 20-60° C. Without wishing to be bound to a particular theory or mechanism, the methodology of the present invention results in a final product with exact chemical structure, and consequently similar biological properties, as the starting material, which allows for simple purification of the product from the reaction reagents.

In some embodiments, the fluorinated compounds comprise PFAr compounds or PFAr derivatives. Said compounds may be according to any one of the following formulas:

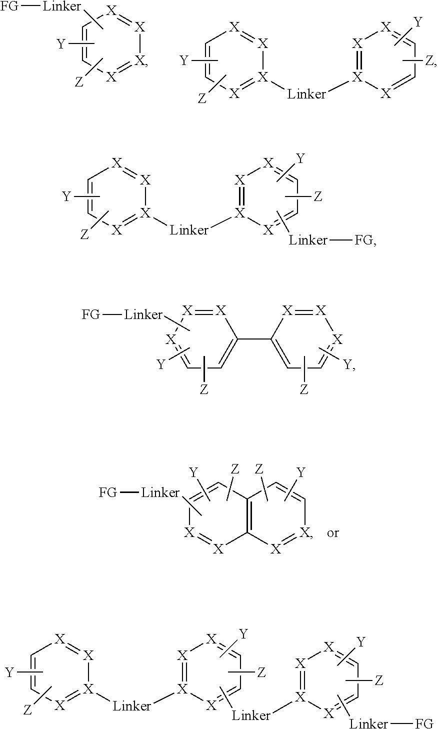

In some embodiments, X is C or N, Y is F (1-6), and Z is Cl, Br, I, NO₂, N₂, N₃, CO—NH₂, SH, SO₃H, COOH, COOR, or NR₃.

In some embodiments, R can be a Methyl, Ethyl, Propyl, Butyl, Pentyl, and/or their isomers. In some embodiments, FG can be maleimide, NHS-ester, azide, tetrazine, alkyne, or alkene.

In some embodiments, the Linker is optional or is SO₂—, SO—, CO—, CO—NH—, —O—, —S—, COO, —(CH₂CH₂O)ₙ—, —(CO-A-NH)ₙ—, —(CO—CH(CHCH₂OH)—NH)ₙ—, —(CO—CH₂—NH)ₙ—, —(CO—CH(CH₃)—NH)ₙ—, —COC₆H₄—, —CH₂CONH—, —NCCCC₆H₄—, —NHCO—, or —NHCS—. In some embodiments, A is —(CH₂)ₙ—. In some embodiments, n ranges from 1 to 10.

In other embodiments, the fluorinated aromatic compound may have poor solubility. The linker may be a water-soluble linker, such as amino or thiol PEG, that can increase the solubility of the fluorinated aromatic compound into a solvent.

Solvent(s) that may be used in the reactions includes, but is not limited to, water, an organic solvent, or a mixture of water and organic solvent. In some embodiments, organic solvents are predominantly solvents with a boiling point lower than 85° C. that is dried under reduced pressure with heating <60° C. upon completion of ¹⁸F-labeling. Non-limiting examples of these organic solvents include ACN, EtOH, MeOH, iPrOH, PrOH, t-BuOH, THF, DEE, DCM, and acetone. In other embodiments, the solvent may comprise up to about 20% of other polar organic solvents with high boiling points, such as, for example, DMF or DMSO.

In some embodiments, examples of the ¹⁸F salt include, but are not limited to, K¹⁸F, Na¹⁸F, Cs¹⁸F, ¹⁸F—K_{2.2.2}/K₂CO₃, crown ethers, or ¹⁸F—NR₄ (where R can be Methyl, Ethyl, Propyl, Butyl, or Pentyl).

Other additive(s) that may be used in the reactions include up to about 5% surfactants such as TPGS-750-M, PTS, SDS, FI-750-M, Pluronic F-127, Tween 20, or Nok. Alternatively or in conjunction, the additives may be up to about 0.5M of organic and/or inorganic salts including, but not limited to, sodium chloride, guanidinium citrate, guanidinium sulfate, guanidinium chloride, guanidinium thiocyanate, ammonium chloride, ammonium citrate, and ammonium sulfate. In other embodiments, a catalyst may be used in the reactions.

Without wishing to be bound to a particular theory or mechanism, the methods of the present invention are advantageous because they do not require extensive purification work up of the radiotracer post radiolabeling. Alternatively, the reaction condition for ¹⁸F-labeling of PFAr allows direct addition of the reaction mixture to conjugate a biomolecule for preparation of ¹⁸F—PFAr-biomolecules.

In some embodiments, the ¹⁸F-labeled radiotracer described herein may be used as a companion diagnostic or companion therapeutic compound for treatment or diagnostic applications.

### A. Conjugation Followed by Radiolabelling

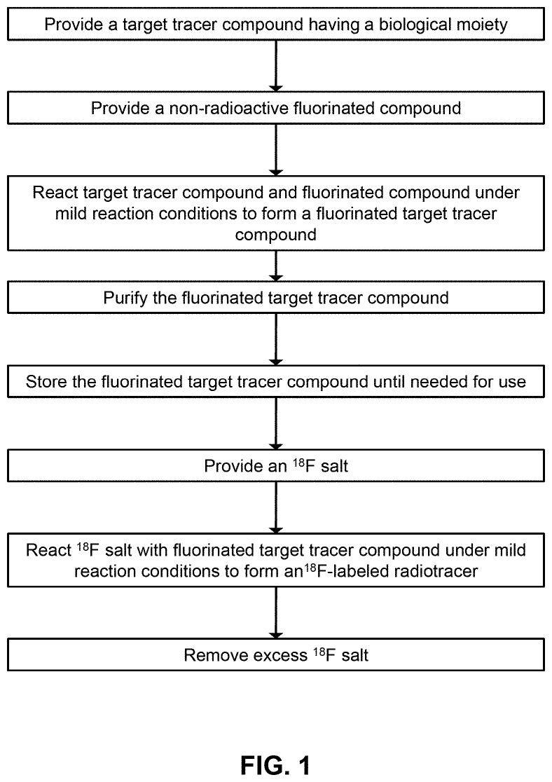

According to some embodiments, the present invention features a method of preparing an ¹⁸F-labeled radiotracer for use in positron emission tomography (PET). Referring to FIG. 1, the method may comprise providing a target tracer compound having a biological moiety, providing a non-radioactive fluorinated compound, reacting the target tracer compound and the fluorinated compound, thereby forming a non-radioactive fluorinated target tracer compound, providing an ¹⁸F salt, and reacting the ¹⁸F salt with the fluorinated target tracer compound, thereby forming the ¹⁸F-labeled radiotracer. In some embodiments, the method may further comprise purifying the fluorinated target tracer compound subsequently after the reaction. In some other embodiments, the method may further comprise removing excess ¹⁸F salt from the second aqueous solvent after the ¹⁸F-labeled radiotracer is formed. For example, the excess ¹⁸F salt can be removed using dialysis or chromatography. Without wishing to limit the invention to a particular theory or mechanism, the method may be effective for preserving a biological activity of the biological moiety.

According to some embodiments, conjugation reactions may be followed by a purification step to isolate the conjugated product from any unconjugated compounds. Without wishing to limit the invention to a particular theory or mechanism, given that the purification of ¹⁸F-labeled materials requires some special conditions and/or equipment, the present invention conveniently performs all chemical reactions and purifications prior to the ¹⁸F/¹⁹F exchange step. In addition, since the radioactive ¹⁸F atom has a half-life of about 109 minutes, it is more beneficial and efficient to perform the ¹⁸F/¹⁹F exchange as the final step, or just prior to the desired time of administering the radiotracer to the subject. Contrary to the present invention, the radioactivity of ¹⁸F decreases to a much greater extent during the longer synthesis and purification steps disclosed in the procedure of Jacobson et al.

In some embodiments, the biomolecule or MRI agent can be conjugated to a cold (i.e. non-radioactive) fluorinated compound and the product may be purified. Since the fluorinated target tracer lacks any radioactivity, the reaction and purification steps can proceed without any urgency or time limitations. In some embodiments, the fluorinated target tracer is stable for a period of time (ca. days to months). Hence, the step of ¹⁸F/¹⁹F exchange may be performed at a later time and at a different location from when and where fluorinated target tracer is prepared.

Further still, a non-chemist or one having only ordinary skill can perform the radiolabelling step of mixing the fluorinated target tracer with an ¹⁸F salt to produce the PET radiotracer, which may have a reaction time as short as 10 minutes. In other embodiments, the PET radiotracer may be ready for use after a simple dialysis step to remove excess ¹⁸F salts. Systems and methods of dialysis are known to one of ordinary skill in the art.

In some embodiments, the target tracer compound may be any molecule that has a biological moiety. In other embodiments, the target tracer compound may be a biomolecule. Non-limiting examples of the target tracer compound include any protein/antibody fragments such as scFv, minibody, diabody, nanobody, and affibody, hormones, antibodies, glycoproteins, peptides, mRNA, siRNA, snRNA, DNA and fragments thereof, carbohydrates, polycarbohydrates, cofactors, coenzymes, phospholipids, glycoproteins, hormones, polyethylene glycols (PEG), PEGylated biologics, PEGylated phospholipids, magnetic resonance imaging (MRI) agents, ultrasound agents, x-ray agents, computerized tomography (CT) agents, fluorescent agents, and synthetic organic or inorganic small molecules.

According to other embodiments, the target tracer compound may further comprise a functional group that reacts with the PFAr compound via aromatic nucleophilic substitution (SNAr). In one embodiment, the functional group may be

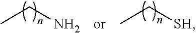

where n ranges from 0-5, or -Ph-OH.

In some embodiments, the step of providing the fluorinated compound may comprise modifying a base fluorinated compound with a water-soluble functional group, thereby increasing a solubility of the PFAr compound to produce a water-soluble fluorinated compound. In some embodiments, the water-soluble functional group is an amino, a thiol, or a thiol PEG group. In one embodiment, the fluorinated compound can be any of the fluorinated compounds disclosed herein. For example, the fluorinated compound may be those shown in FIG. 3 with n ranging from 2-8 or those according to the aforementioned formulas.

In preferred embodiments, the target tracer compound and the fluorinated compound are reacted in a first aqueous solvent at a first ambient temperature that is mild for the biological moiety such that the biological activity of the biological moiety is preserved. In one embodiment, the first aqueous solvent is predominantly water. In another embodiment, the first aqueous solvent may further comprise a base. The base of the first aqueous solvent may be effective for increasing the nucleophilicity of the target tracer compound. Examples of said base include, but are not limited to, tris(hydroxymethyl)aminomethane, phosphate, diisopropylethylamine, and 4-(2-hydroxyethyl)piperazine-1-ethanesulfonic acid. In some embodiments, the base is present at a range of about 1%-5% vol, or about 5%-10% vol, or about 10%-15% vol, or about 15%-20% vol, including any ranges in between said values.

In another embodiment, the first aqueous solvent may further comprise about 1%-10% vol of a co-solvent. Non-limiting examples of the co-solvent include dimethyl sulfoxide, dimethylformamide, acetonitrile, EtOH, MeOH, iPrOH, PrOH, t-BuOH, THF, DEE, DCM, and acetone. Preferably, the co-solvent may be effective for increasing a solubility of the target tracer compound. In some embodiments, the co-solvent is present at a range of about 1%-4% vol, or about 4%-7% vol, or about 7%-10% vol, including any ranges in between said values. In a preferred embodiment, the amount of the co-solvent is up to about 5% vol.

In some embodiments, the first ambient temperature can range from about 15° C. to about 60° C., including any ranges in between said values. For example, the first ambient temperature is about 15-20° C., or about 20-40° C., or about 40-60° C., including any ranges in between said values. In other embodiments, the first ambient temperature is at most about 60° C.

In some embodiments, prior to reacting the fluorinated target tracer with the ¹⁸F salt, the fluorinated target tracer compound can be stored for a period of time until an ¹⁸F-labeled radiotracer is required for use in PET. For instance, the fluorinated target tracer compound may be stored for a period of time ranging from days to months. When the ¹⁸F-labeled radiotracer is required for PET, the stored fluorinated target tracer compound is reacted with the ¹⁸F salt to form the ¹⁸F-labeled radiotracer.

In other preferred embodiments, the ¹⁸F salt is reacted with the fluorinated target tracer compound in a second aqueous solvent at a second ambient temperature that is mild for the biological moiety such that the biological activity of the biological moiety is preserved. Non-limiting examples of the ¹⁸F salt include Na¹⁸F, K¹⁸F, or K¹⁸FK_{2.2.2}. Without wishing to limit the invention to a particular theory or mechanism, the ¹⁸F-labeled radiotracer is formed when an ¹⁸F radioisotope of the ¹⁸F salt replaces any of the ¹⁹F isotopes in the fluorinated target tracer compound. Preferably, the ¹⁸F-labeled radiotracer is formed in about 10 to 20 minutes; for example, in about 15 minutes.

In some embodiments, the second aqueous solvent may be predominantly water. In other embodiments, the second aqueous solvent may further comprise about 1%-10% vol of a co-solvent that is effective for increasing a solubility of the fluorinated target tracer compound. Examples of the co-solvent include, but are not limited to, dimethyl sulfoxide, dimethylformamide, acetonitrile, EtOH, MeOH, iPrOH, PrOH, t-BuOH, THF, DEE, DCM, and acetone. In still other embodiments, the co-solvent is present at a range of about 1%-4% vol, or about 4%-7% vol, or about 7%-10% vol, including any ranges in between said values. In a preferred embodiment, the amount of the co-solvent is up to about 5% vol.

In one embodiment, the second ambient temperature can range from about 15° C. to about 60° C., including any ranges in between said values. For example, the second ambient temperature is about 15-20° C., or about 20-40° C., or about 40-60° C., including any ranges in between said values. In another embodiment, the second ambient temperature is at most about 60° C.

In other embodiments, additive(s) such as surfactants and/or organic and/or inorganic salts may be used in any of the reaction steps. In one embodiment, the additive may comprise up to 5% surfactants. For example, the additive may be about 0.1%-1% surfactant, about 1%-3% surfactant, or about 3%-5% surfactant. In another embodiment, the additive may comprise up to 0.5M organic and/or inorganic salts. For example, the additive may be about 0.01M-0.1M organic and/or inorganic salts, about 0.1M-0.3M organic and/or inorganic salts, or about 0.3M-0.5M organic and/or inorganic salts.

According to some embodiments, the present invention features a kit for preparing an ¹⁸F-labeled radiotracer for use in positron emission tomography (PET). In one embodiment, the kit may comprise a fluorinated target tracer compound, an ¹⁸F salt, and a set of instructions for preparing the ¹⁸F-labeled radiotracer prior to use in PET such that the biological activity of the biological moiety is preserved. In some embodiments, the fluorinated target tracer compound may comprise a fluorinated compound covalently bound to a target tracer compound having a biological moiety in which its biological activity is preserved. Preferably, the fluorinated target tracer compound is non-radioactive. In another embodiment, the fluorinated target tracer compound may be a purified form.

In some embodiments, the set of instructions may comprise an instruction for reacting the ¹⁸F salt with the fluorinated target tracer compound in an aqueous solvent at an ambient temperature that is mild for the biological moiety such that its biological activity is preserved. During the reaction, an ¹⁸F radioisotope of the ¹⁸F salt is configured to replace an ¹⁹F isotope of the fluorinated target tracer compound, thereby forming the ¹⁸F-labeled radiotracer. In other embodiments, the set of instructions may further comprise an instruction for removing excess ¹⁸F salt after the ¹⁸F-labeled radiotracer is formed.

In other embodiments, the kit may further comprise additive(s) such as surfactants and/or organic and/or inorganic salts.

### B. Radiolabelling Followed by Conjugation

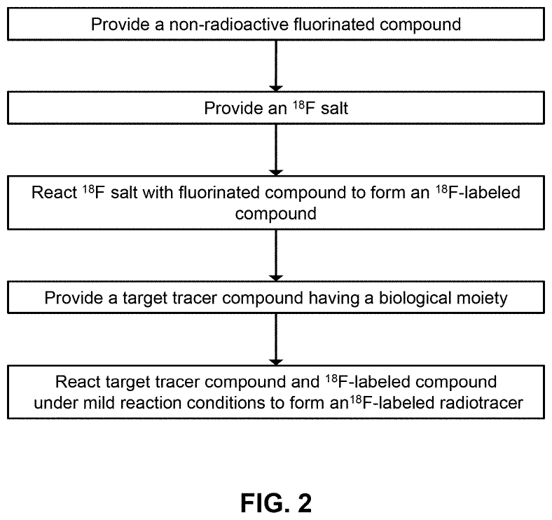

According to other embodiments, the present invention features a method of preparing an ¹⁸F-labeled radiotracer. Referring to FIG. 2, the method may comprise preparing an ¹⁸F-labeled compound, providing a target tracer compound having a biological moiety, and reacting the target tracer compound and the ¹⁸F-labeled compound, thereby forming the ¹⁸F-labeled radiotracer. Without wishing to limit the invention to a particular theory or mechanism, the method may be effective for preserving a biological activity of the biological moiety. Furthermore, this method will save time because it requires no purification.

In some embodiments, the step of preparing the ¹⁸F-labeled compound may comprise conjugating a non-radioactive fluorinated compound to a linker with an active functional group, providing an ¹⁸F salt, and reacting the ¹⁸F salt with the non-radioactive fluorinated compound that is conjugated to the linker such that said fluorinated compound is ¹⁸F-labeled, thereby forming the ¹⁸F-labeled compound.

In other embodiments, the step of preparing the ¹⁸F-labeled compound may comprise providing an ¹⁸F salt, and reacting the ¹⁸F salt with the non-radioactive fluorinated compound such that said fluorinated compound is ¹⁸F-labeled, thereby forming the ¹⁸F-labeled compound. In one embodiment, the non-radioactive fluorinated compound may already have a functional group that can act as a linker for direct or indirect conjugation to the target tracer compound, i.e., a biomolecule. Alternatively, the target tracer compound may further comprise a functional group that can react with the ¹⁸F-labeled compound via aromatic nucleophilic substitution (SNAr). In one embodiment, the functional group may be

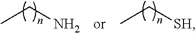

where n ranges from 0-5.

The non-radioactive fluorinated compound can be any of the fluorinated compounds disclosed herein. For example, the fluorinated compound may be those shown in FIG. 3 with n ranging from 2-8, or those according to the aforementioned formulas.

In some embodiments, the non-radioactive fluorinated compound may comprise or be modified with a water-soluble functional group, thereby increasing a solubility of the fluorinated compound to produce a water-soluble fluorinated compound. In some embodiments, the water-soluble functional group is an amino, a thiol, or a thiol PEG group.

According to some embodiments, the labeling reaction may be followed by a purification step, which is prior to reacting it with the target tracer compound, to isolate the ¹⁸F-labeled compound from excess ¹⁸F salt. In some embodiments, the step may comprise removing excess ¹⁸F salt. For example, the excess ¹⁸F salt can be removed using dialysis or chromatography.

In some embodiments, the target tracer compound may be any molecule that has a biological moiety. In other embodiments, the target tracer compound may be a biomolecule. Non-limiting examples of the target tracer compound include any protein/antibody fragments such as scFv, minibody, diabody, nanobody, and affibody, hormones, antibodies, glycoproteins, peptides, mRNA, siRNA, snRNA, DNA and fragments thereof, carbohydrates, polycarbohydrates, cofactors, coenzymes, phospholipids, glycoproteins, hormones, polyethylene glycols (PEG), PEGylated biologics, PEGylated phospholipids, magnetic resonance imaging (MRI) agents, ultrasound agents, x-ray agents, computerized tomography (CT) agents, fluorescent agents, and synthetic organic or inorganic small molecules.

In some preferred embodiments, the ¹⁸F salt is reacted with the non-radioactive fluorinated compound in a first solvent and at a first temperature. Non-limiting examples of the ¹⁸F salt include K¹⁸F, Na¹⁸F, Cs¹⁸F, ¹⁸F—K_{2.2.2}/K₂CO₃, crown ethers, or ¹⁸F—NR₄ (where R can be Methyl, Ethyl, Propyl, Butyl, or Pentyl). Without wishing to limit the invention to a particular theory or mechanism, the ¹⁸F-labeled compound is formed when an ¹⁸F radioisotope of the ¹⁸F salt replaces any of the ¹⁹F isotopes in the non-radioactive fluorinated compound. Preferably, the ¹⁸F-labeled compound is formed in about 5 to 20 minutes; for example, in about 15 minutes.

In some embodiments, the first solvent may comprise an organic solvent. Examples of the organic solvent include, but are not limited to, acetonitrile, EtOH, MeOH, iPrOH, PrOH, t-BuOH, THF, DEE, DCM, and acetone. In other embodiments, the first solvent may further comprise about 1%-10% vol of a co-solvent such as dimethyl sulfoxide or dimethylformamide.

In some embodiments, the first temperature can range from about 15° C. to about 60° C., including any ranges in between said values. For example, the first temperature is about 15-20° C., or about 20-40° C., or about 40-60° C., including any ranges in between said values. In another embodiment, the first temperature is at most 60° C.

In other embodiments, the step of preparing the ¹⁸F-labeled compound may further comprise drying the ¹⁸F-labeled compound upon completion of ¹⁸F-labeling to evaporate the first solvent. In one embodiment, the drying is done under reduced pressure with heating up to 60° C. Reduced pressure refers to less than 1 atm.

In other preferred embodiments, the ¹⁸F-labeled compound and the target tracer compound are reacted in a second solvent at a second temperature that is mild for the biological moiety such that the biological activity of the biological moiety is preserved. In one embodiment, the second solvent is predominantly water.

In some embodiments, the second solvent may further comprise about 1%-10% vol of a co-solvent. Non-limiting examples of the co-solvent include dimethyl sulfoxide, dimethylformamide, acetonitrile, EtOH, MeOH, iPrOH, PrOH, t-BuOH, THF, DEE, DCM, and acetone. Preferably, the co-solvent may be effective for increasing a solubility of the target tracer compound and/or the ¹⁸F-labeled compound. In some embodiments, the co-solvent is present at a range of about 1%-4% vol, or about 4%-7% vol, or about 7%-10% vol, including any ranges in between said values. In a preferred embodiment, the amount of the co-solvent is up to about 5% vol.

In another embodiment, the second solvent may further comprise a base. The base of the second solvent may be effective for increasing the nucleophilicity of the target tracer compound. Examples of said base include, but are not limited to, tris(hydroxymethyl)aminomethane, phosphate, diisopropylethylamine, and 4-(2-hydroxyethyl)piperazine-1-ethanesulfonic acid. In some embodiments, the base is present at a range of about 1%-5% vol, or about 5%-10% vol, or about 10%-15% vol, or about 15%-20% vol, including any ranges in between said values.

In some embodiments, the second temperature can range from about 15° C. to about 60° C., including any ranges in between said values. For example, the second temperature is about 15-20° C., or about 20-40° C., or about 40-60° C., including any ranges in between said values. In other embodiments, the first ambient temperature is at most about 60° C.

In other embodiments, additive(s) such as surfactants and/or organic and/or inorganic salts may be used in any of the reaction steps. In one embodiment, the additive may comprise up to 5% surfactants. For example, the additive may be about 0.1%-1% surfactant, about 1%-3% surfactant, or about 3%-5% surfactant. In another embodiment, the additive may comprise up to 0.5M organic and/or inorganic salts. For example, the additive may be about 0.01M-0.1M organic and/or inorganic salts, about 0.1M-0.3M organic and/or inorganic salts, or about 0.3M-0.5M organic and/or inorganic salts.

According to some embodiments, the present invention features a kit for preparing an ¹⁸F-labeled radiotracer. In one embodiment, the kit may comprise a target tracer compound, a non-radioactive fluorinated compound, an ¹⁸F salt, and a set of instructions for preparing the ¹⁸F-labeled radiotracer such that the biological activity of the biological moiety is preserved. In some embodiments, the kit may further comprise a linker compound.

In some embodiments, the set of instructions may comprise an instruction for preparing an ¹⁸F-labeled compound using at least the non-radioactive fluorinated compound and ¹⁸F salt. During the reaction, an ¹⁸F radioisotope of the ¹⁸F salt is configured to replace an ¹⁹F isotope of the non-radioactive fluorinated compound, thereby forming the ¹⁸F-labeled compound. In other embodiments, the set of instructions may further comprise an instruction for reacting the target tracer compound and the ¹⁸F-labeled compound in conditions that preserves the biological activity of the biological moiety, thereby forming the ¹⁸F-labeled radiotracer.

In further embodiments, the kit may further comprise additive(s) such as surfactants and/or organic and/or inorganic salts.

In conjunction with any of the embodiments described herein, the present invention may be used with microfluidic devices to prepare ¹⁸F-labeled biomolecules efficiently.

### EXAMPLES

The following are non-limiting examples of preparing an ¹⁸F-radiolabeled biomolecule in accordance with the present invention. The examples are for illustrative purposes only and are not intended to limit the invention in any way. Equivalents or substitutes are within the scope of the invention.

The following examples investigated the activity of: 1) different types of PFAr with tendencies for a faster ¹⁸F-labeling reaction, 2) organic solvent(s) with easy purification process, 3) type and amount of phase-transfer catalysts/bases and their ratio, and 4) temperature range.

Example 1: An MRI agent or a biomolecule is conjugated to PFAr in an aqueous solution under mild conditions, as shown in Scheme 1.

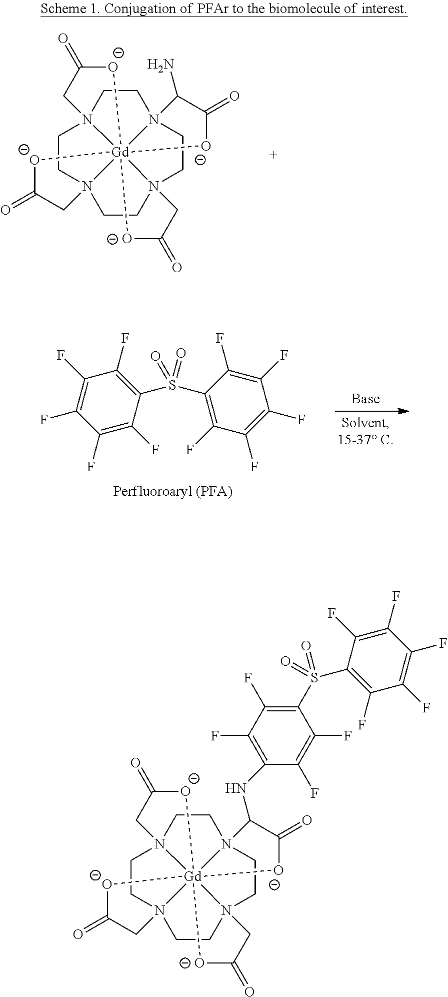

In some embodiments, water may be the main solvent for this reaction. However, other co-solvents, such as DMSO, DMF, and ACN (up to 5%), may be added to the water if the MRI agent, biomolecule, or PFAr has poor solubility in water. In other embodiments, the base may be TRIS, phosphate, DIPEA, or HEPES. Preferably, the base may be effective to increase the nucleophilicity of the MRI agent or biomolecule. In preferred embodiments, the reaction may be performed at a mild temperature range for the biomolecule. For example, the temperature can range from about 15-37° C.

Example 2: ¹⁸F salts are added to the solution of PFA-conjugated MRI agent or biomolecule, as shown in Scheme 2. ¹⁸F/¹⁹F exchange can occur rapidly in this step.

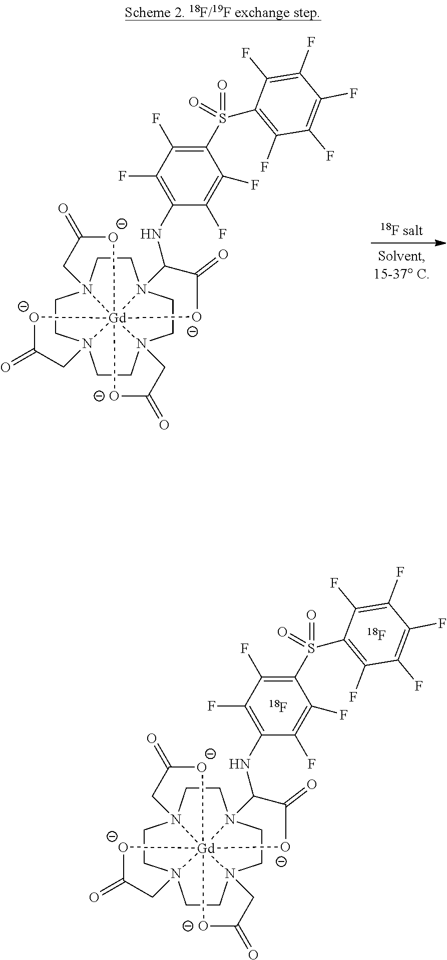

In some embodiments, water may be the main solvent for the reaction. However, other co-solvents, such as DMSO, DMF, and ACN (up to 5%), may be added to the water if the MRI agent, biomolecule, or PFAr has poor solubility in water. In other embodiments, the ¹⁸F salt may be Na¹⁸F, K¹⁸FK_{2.2.2}, or similar compounds. This reaction is also performed at a mild temperature range for the biomolecule. In one embodiment, the temperature can range from about 15-37° C. Without wishing to limit the invention to a particular theory or mechanism, the present methodology advantageously utilizes reaction conditions that are harmless for biomolecules, thereby retaining their biological activity.

Example 3: Scheme 3 shows another non-limiting example of the reaction procedure. The MRI agent or a biomolecule is conjugated to PFAr in an aqueous solution under mild conditions, and then ¹⁸F salts are added to the solution of the PFA-conjugated MRI agent or biomolecule, thereby producing the ¹⁸F radiolabeled MRI agent or biomolecule.

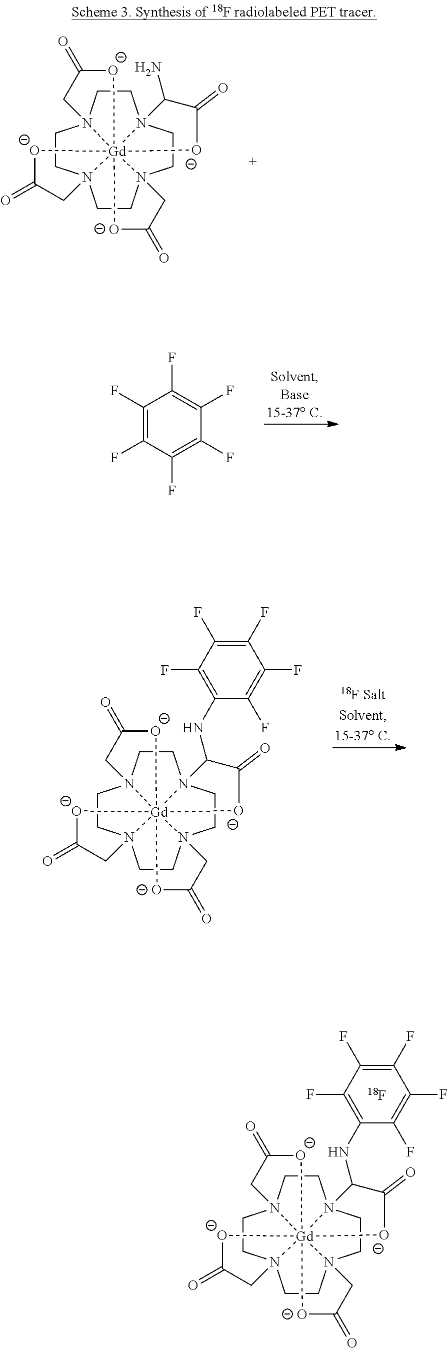

Example 4: Duramycin, a cyclic peptide that is conjugated to a PFAr is labeled with 18F in mild a condition, as shown in Scheme 4.

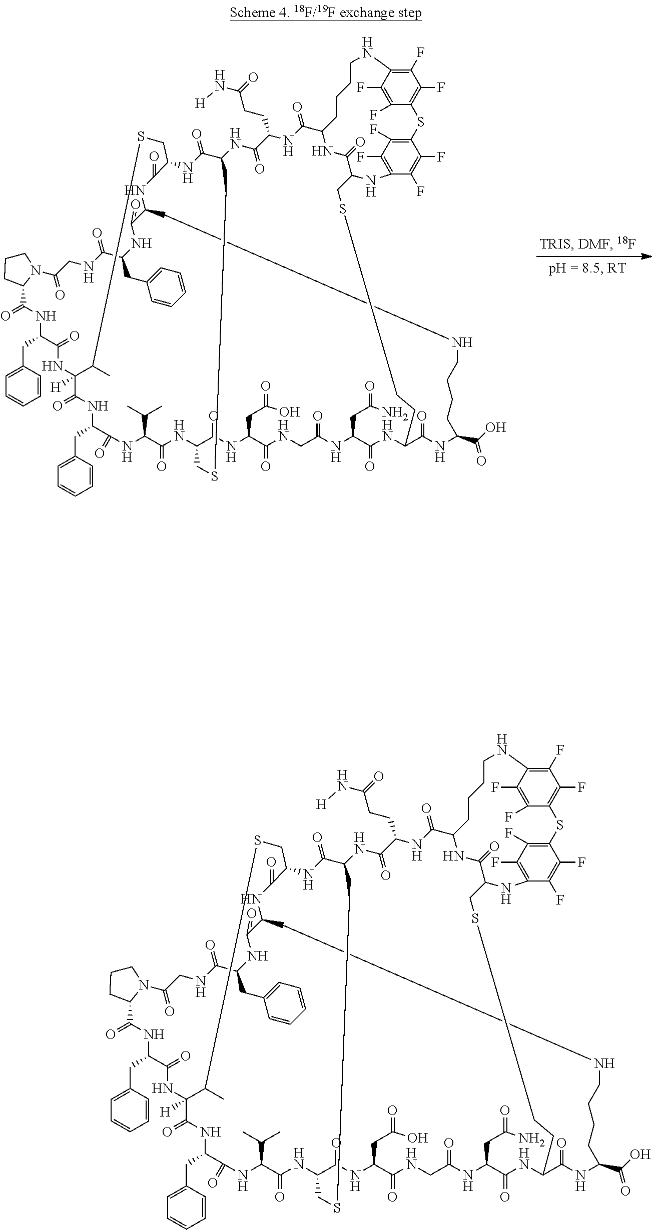

In some embodiments, water may be the main solvent for the reaction. However, other co-solvents, such as DMSO, DMF, and ACN, may be added to the water if the biomolecule has poor solubility in water. In other embodiments, the ¹⁸F salt may be K¹⁸F, Na¹⁸F, Cs¹⁸F, ¹⁸F—K_{2.2.2}/K₂CO₃, and crown ethers, or ¹⁸F—NR₄ (where R can be Methyl, Ethyl, Propyl, Butyl, or Pentyl). This reaction is also performed at a mild temperature range for the biomolecule. In one embodiment, the temperature can range from about 15-40° C. Without wishing to limit the invention to a particular theory or mechanism, the present methodology advantageously utilizes reaction conditions that are harmless for biomolecules, thereby retaining their biological activity.

### Example 5: Pentafluoropyridine (PFPy) Labeling for Optimization

PFPy is a base that can readily react with other chemical moieties such as carboxylic acid, amine, and thiol groups. In addition, it has suitable water solubility to interact with more fluoride ions in water. To introduce a process rival to the current ¹⁸F-prosthetic groups, the focus was on decreasing the amount of starting PFAr carrying ¹⁹F so the final ¹⁸F-labeled had a large specific (molar) activity. While reactions proceeded in other organic solvents, acetonitrile was the solvent of choice because it can be tolerated by sensitive biomolecules for bioconjugation reactions (in contrast to DMSO or DMF) and it mixes with water easily. Furthermore, it is compatible with post-synthesis procedure for purification of the ¹⁸F-labeled biomolecules (e.g. spin dialysis columns can tolerate up to 20% of ACN but only up to 5% of DMSO, or a ¹⁸F-labeled biomolecule in 100% ACN can be directly injected to a HPLC for purification without harming the system). A temperature range between room temperature to 60° C. and a 0.1X-8X equivalent ratio (between PFPy and the base/PTC) showed the most efficient ¹⁸F-labeling. Presence of an auxiliary base and a PTC, [quaternary amine(s)] led to higher yield and specific (molar) activities of the labeled products. Increasing the equivalent amount of the base/PTC led to a lower labeling yield. In some cases, addition of organic and inorganic salts increased the radiolabeling yield and shortened the reaction time.

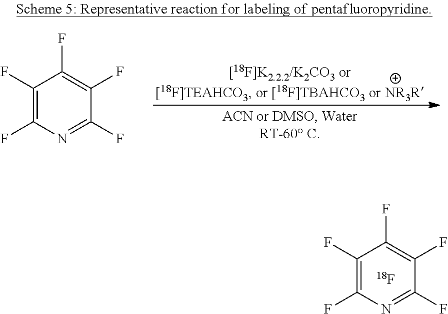

A very small amount of PFPy (a range between 0.001-100 μmol) was used to increase specific/molar activity. To compensate for the low starting amount of the PFPy, the amount of organic solvent was decreased to have a concentration in a range between 0.05 mM to 400 mM. Since fluoride ion precipitates out in such a small amount of organic solvent (even in presence of PTC), water was kept up to 50% (v/v) in the reaction vessel. In addition, a reaction temperature up to 60° increased solubility of the fluoride ions in the reaction mixture. Upon completion of the reaction, separation of the ¹⁸F—PFAr was performed using common chromatography columns/cartridges with C18, C8, C3 resins (or other common purification columns/cartridges) by a water wash followed by a wash using a mixture of water and an organic solvent.

In some instances where quaternary amines on resins were used (instead of a phase-transfer catalyst/base system), an alternative approach was utilized. The alternative approach required a simple wash of ¹⁸F—PFAr from the resin. Then a portion of ¹⁸F—PFAr in acetonitrile (or other solvents) was directly added to a solution of the biomolecule in aqueous (buffer) media or organic solvent for bioconjugation. In some cases, a bioconjugation handle, such as azide, NHS ester, maleimide, or similar bioconjugation handles with or without a linker, was introduced. In case of some biomolecules, bioconjugation was performed first, and then performed ¹⁸F/¹⁹F exchange under the same mild condition where the biomolecule was stable and retained its biological function.

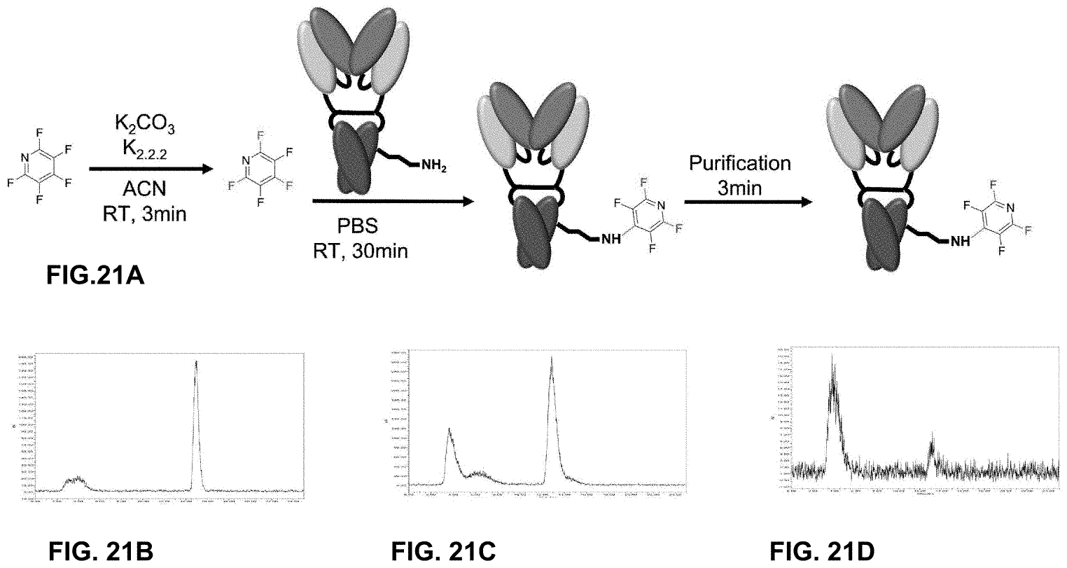

Example 6: Referring to FIG. 21A-21D, the method of present invention allows for design and preparation of ¹⁸F—PFAr-labeled antibody fragments. Pentafluoropyridine that is labeled with ¹⁸F in acetonitrile (up to 20%) is added to antibody fragments in a buffer (e.g., PBS) in presence of a base. Examples of antibody fragments include, but are not limited to, minibodies, diabodies, cys-diabodies, scFv, affibodies, and nanobodies.

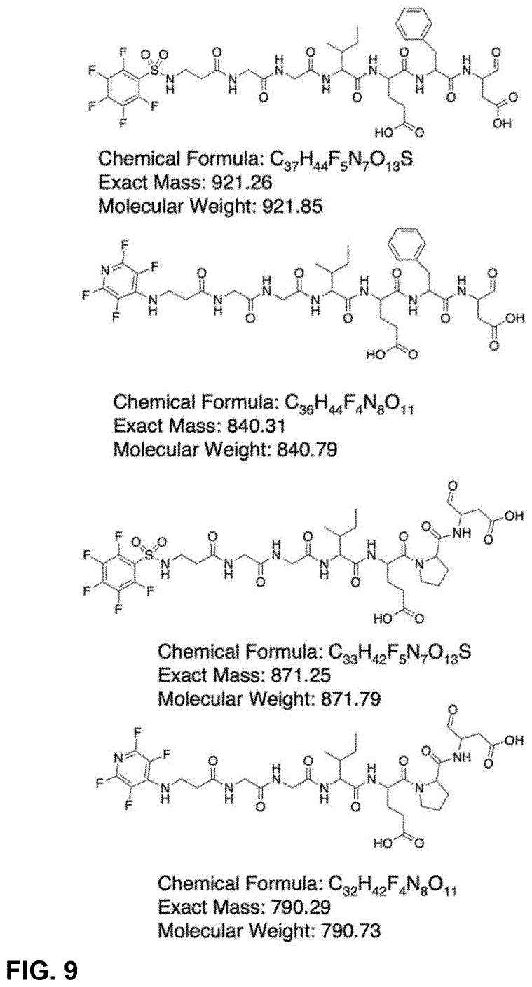

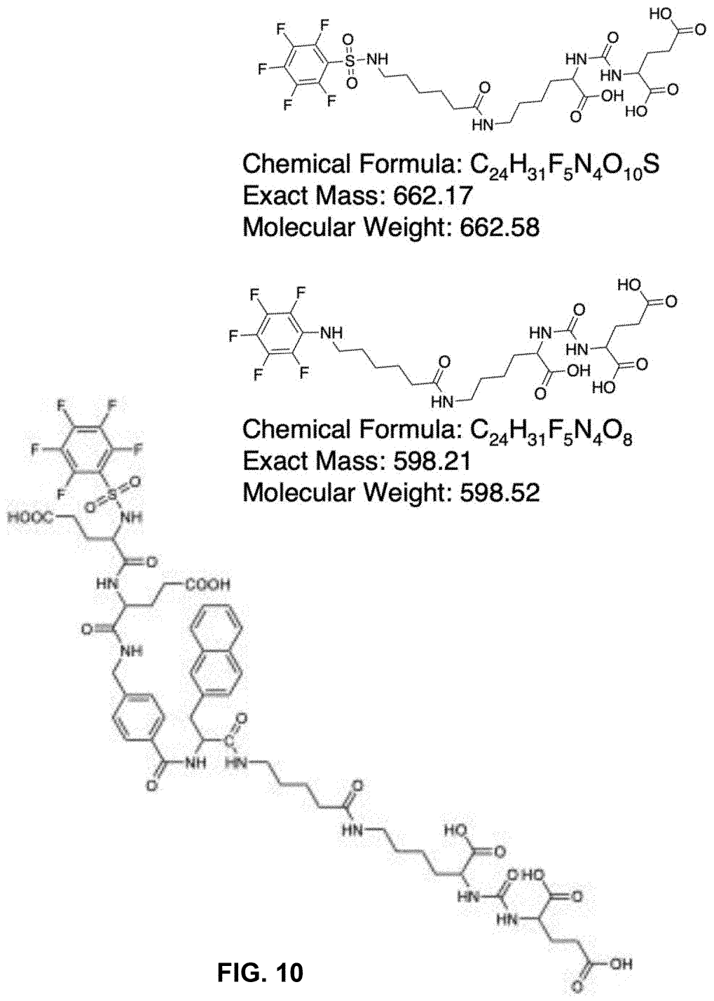

Example 7: Referring to FIG. 9, the method of the present invention allows for design and preparation of radiotracers, as irreversible inhibitors, for detection and quantification of proteases with active functional groups. Such radiotracers are conjugated to fluorinated aromatic compounds first and then are ¹⁸F-labeled with this methodology. Examples of proteases include, but are not limited to, cysteine proteases, serine proteases, aspartic proteases, and metalloproteases. The active functional groups may be tetrafluoro phenol, 2,6-dimethylbenzoate, aldehyde, and their derivatives.

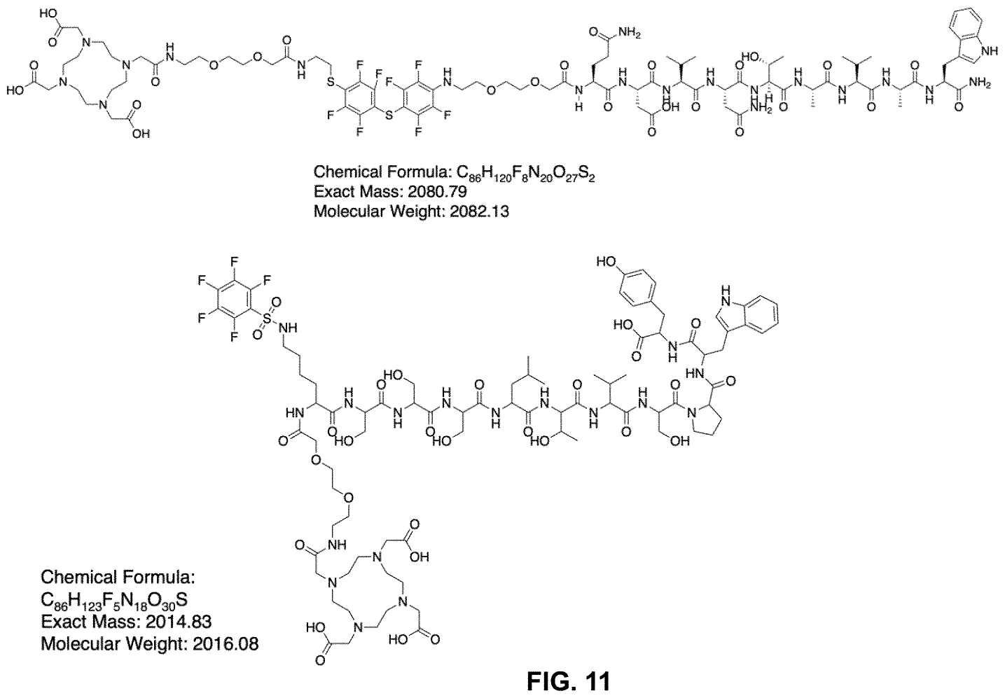

Example 8: Referring to FIG. 11, the method of the present invention allows for design and preparation of diagnostic pairs for radiotherapeutics (e.g. biologics with a chelate for Alpha/Beta Emitters) where both diagnostic and therapeutic pairs have the same chemical and biological properties. Compared to late-stage ¹⁸F-labeling of such therapeutic compounds that require extensive purification and characterization, the methodology of the present invention benefits from a short and simple purification procedure.

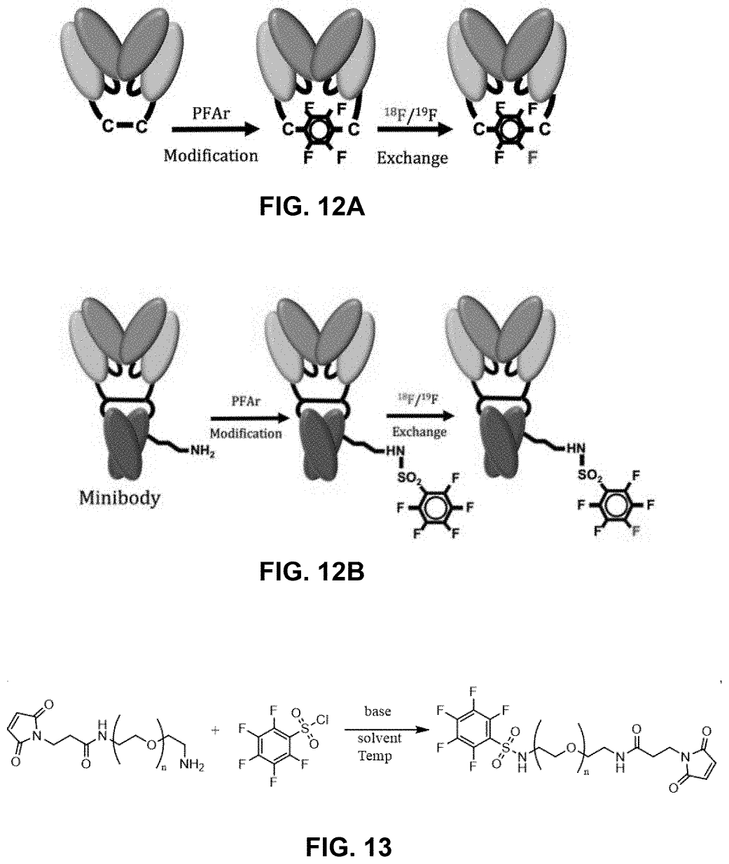

Example 9: Referring to FIGS. 12A-12B, antibody fragments may be conjugated to fluorinated compounds via direction reaction on the aromatic ring of the fluorinated compound through the side chains of amino acids (e.g., cysteine, lysine, arginine, tryptophane, tyrosine, serine, threonine, aspartic acid, or glutamic acid) or unnatural amino acids, and then the exchange reaction is performed on the conjugated product.

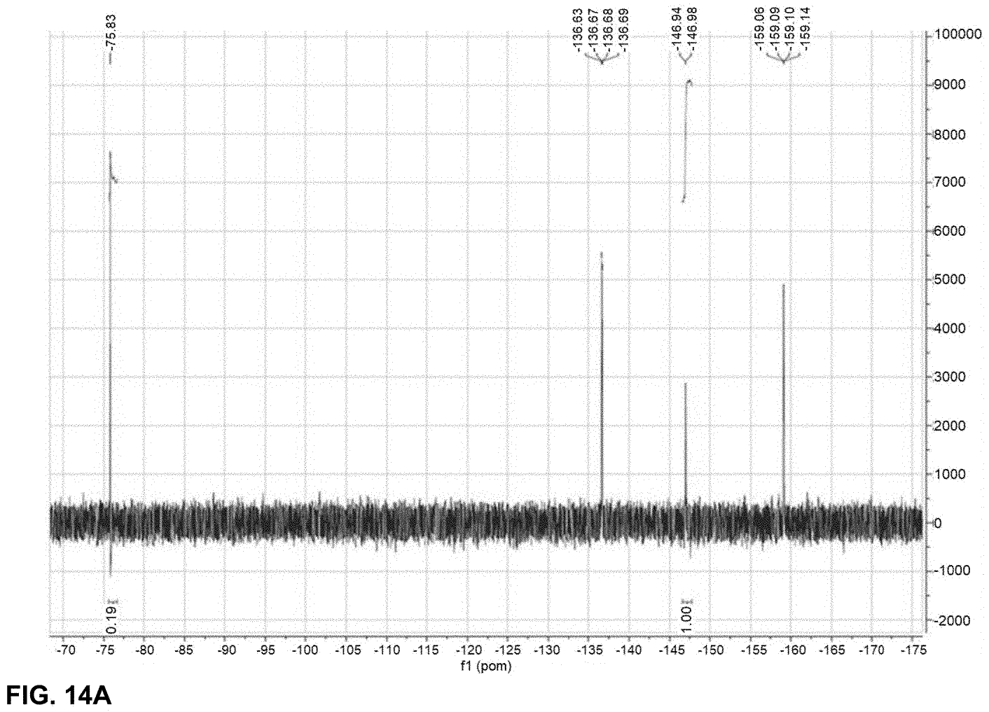

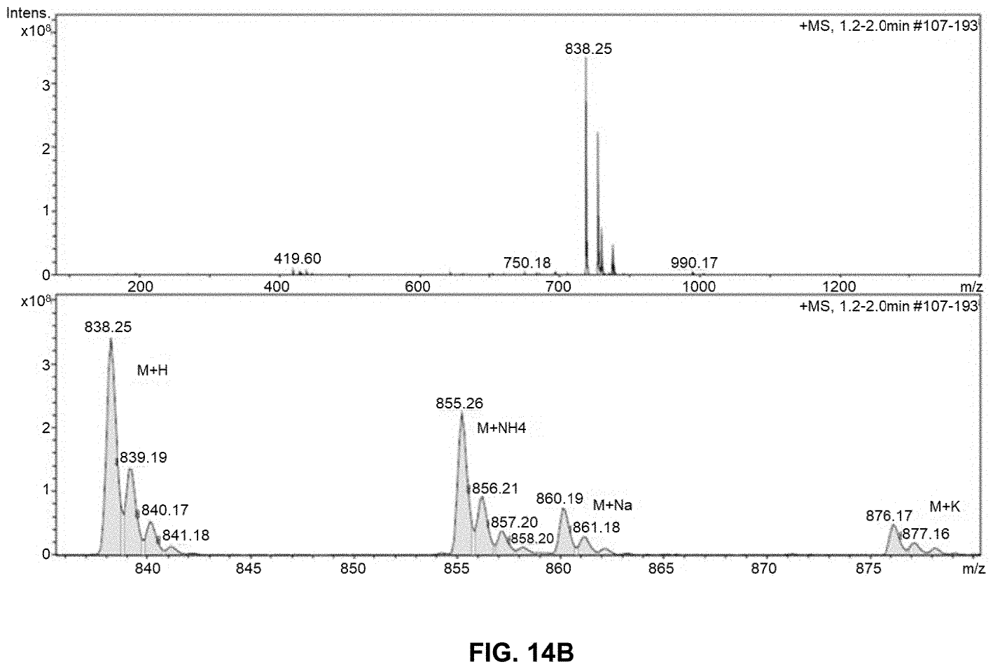

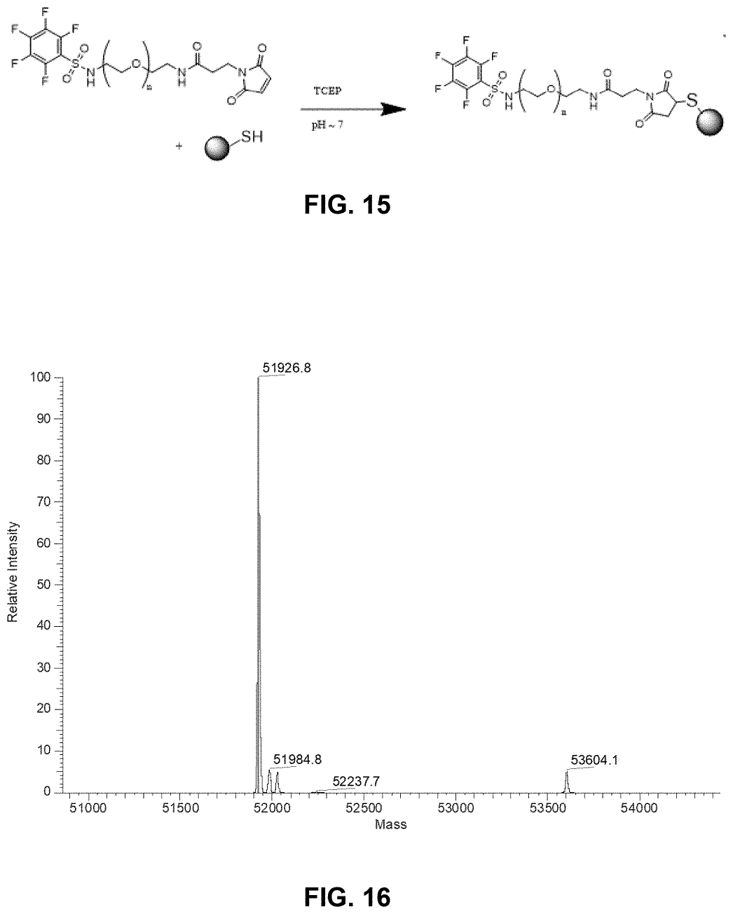

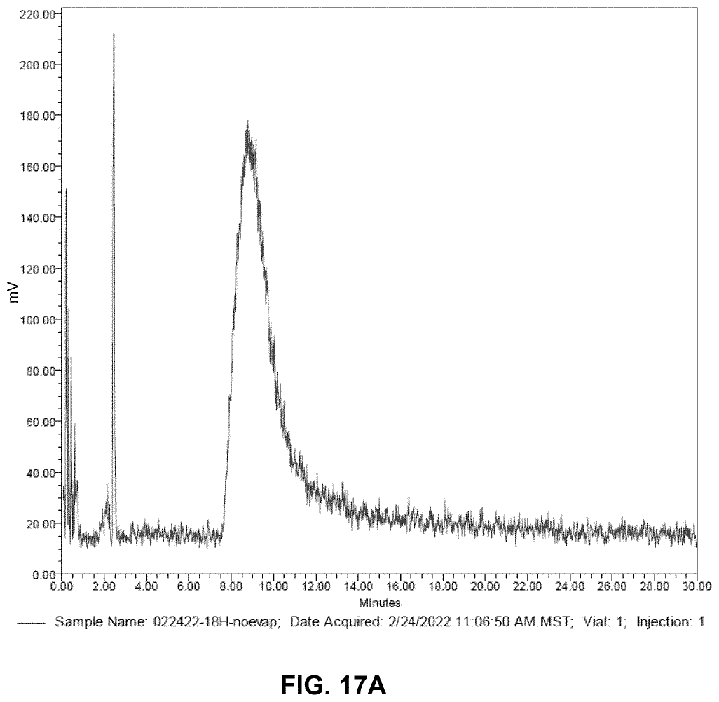

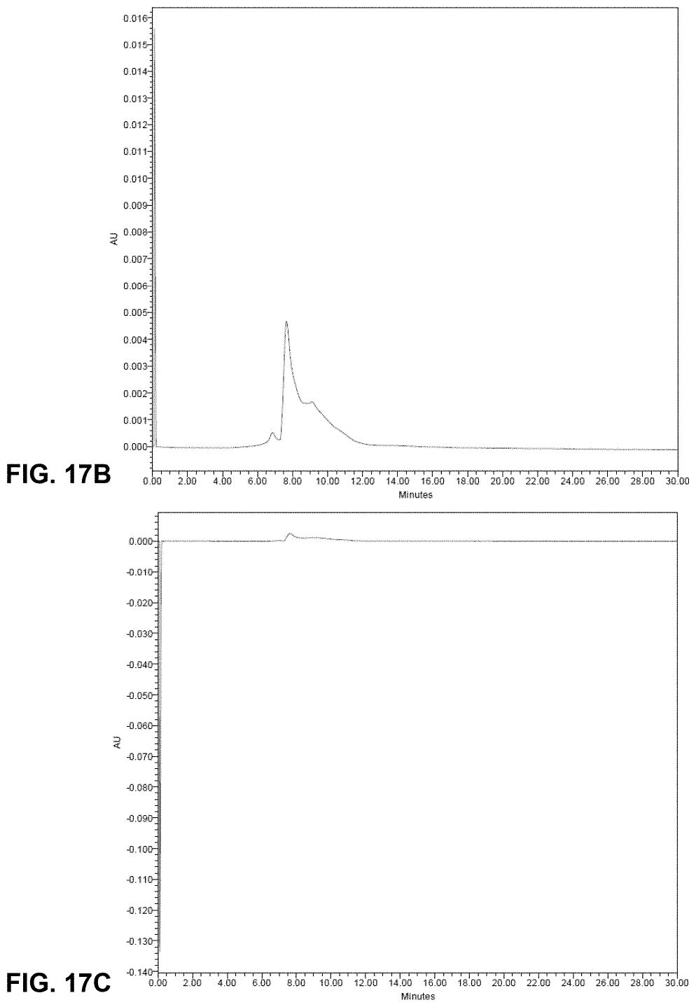

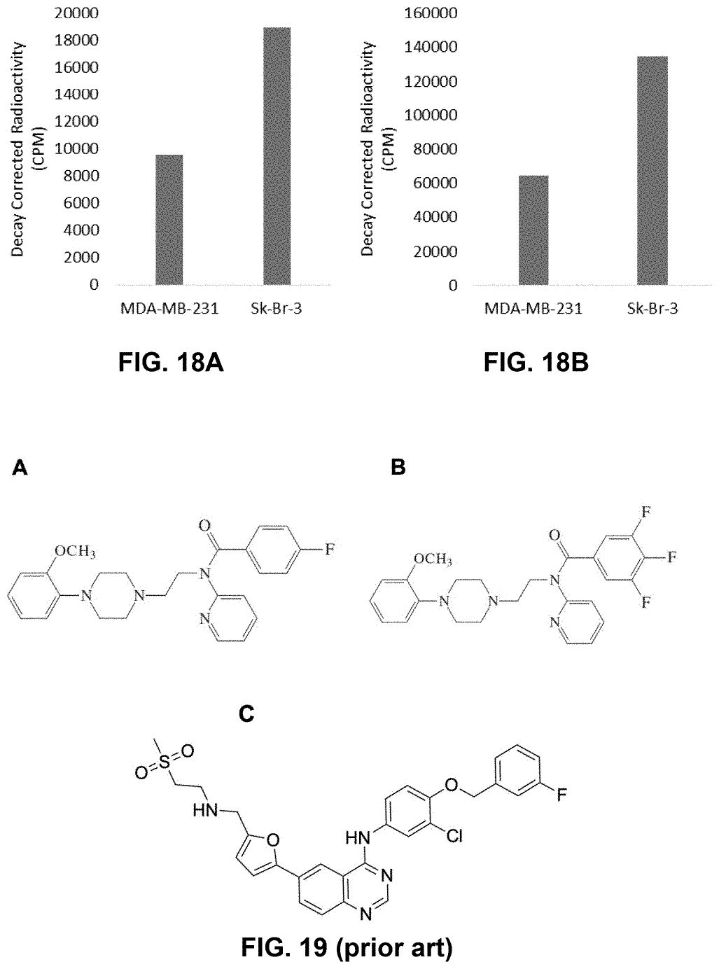

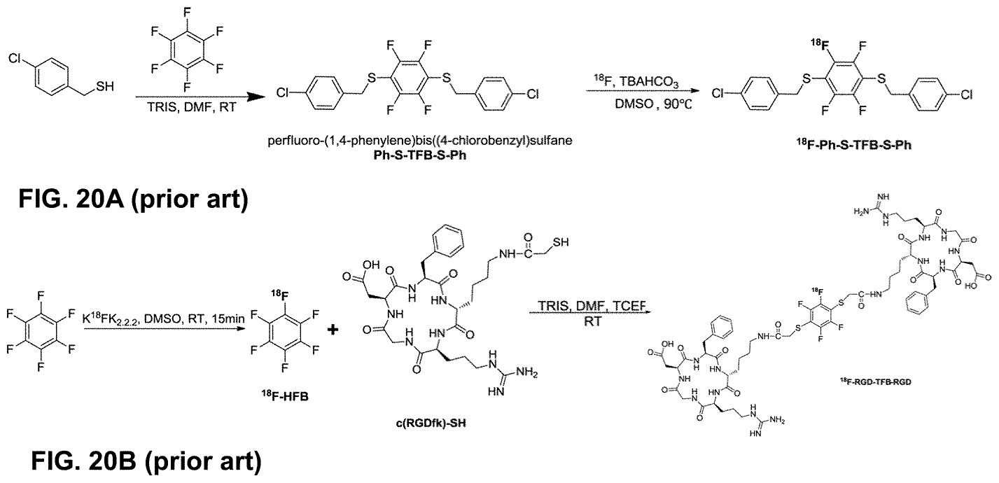

Example 10: Referring to FIGS. 13-18B, the radiolabelling step is done prior to conjugation with a biomolecule. In FIG. 13, a fluorinated aromatic compound is conjugated to a linker with an active functional group for direct or indirect conjugation to the biomolecule. The conjugated fluorinated aromatic compound is then ¹⁸F-labeled in an aqueous solution. In FIG. 15, the ¹⁸F-labeled fluorinated aromatic compound conjugated to a linker is then conjugated to the biomolecule. Mild reaction conditions allowed for the ¹⁸F-labeled biomolecule to retain its biological properties as shown in FIGS. 18A-18B.

Example 11: ¹⁸F-labeling was performed on a sub micro-mol amount of pentafluoropyridine precursor using 2-5 mCi of ¹⁸F water manually to obtain improved molar/specific activity (15-20 mCi/umol). Automating this process or starting with higher ¹⁸F activity will also significantly improve the molar/specific activity.

Example 12: ACN was used as a solvent of choice because it is more compatible with sensitive biomolecules. Using up to 20% acetonitrile, the inventors were able to conjugate ¹⁸F-tetrafluorpyridine to antibody fragments in aqueous buffers (e.g. PBS) without any concern for post-conjugation purification of ¹⁸F—PFAr-biomolecules with size exclusion chromatography/or spin dialysis column. Unlike ACN, the inventors could not use more than 5% DMSO for the same process. Otherwise, the size exclusion chromatography/or spin dialysis column could not be used for purification.

As used herein, the term “about” refers to plus or minus 10% of the referenced number.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A method of preparing an ¹⁸F-labeled radiotracer for use in positron emission tomography (PET), said method comprising:
a) providing an ¹⁸F compound;
b) providing a target tracer compound having a biological moiety; and
c) reacting the ¹⁸F compound with the target tracer compound in a solvent and at a temperature that is mild for the biological moiety, thereby forming the ¹⁸F-labeled radiotracer, wherein the biological activity of the biological moiety is preserved.

2. The method of claim 1, wherein providing a target tracer compound having a biological moiety comprises conjugation, and reacting the ¹⁸F compound with the target tracer compound comprises radiolabeling.

3. The method of claim 2, wherein the ¹⁸F compound is an ¹⁸F salt.

4. The method of claim 3, wherein providing the target tracer compound having the biological moiety comprises:
reacting the target tracer compound with a non-radioactive fluorinated compound in an solvent that is predominantly water, at a temperature that is mild for the biological moiety, thereby forming a fluorinated target tracer compound, wherein the fluorinated target tracer compound is non-radioactive, and wherein a biological activity of the biological moiety is preserved.

5. The method of claim 4, wherein the temperature is at most 60° C.

6. The method of claim 4, wherein the solvent in which the ¹⁸F compound is reacted with the target tracer compound comprises a second solvent that is predominantly water.

7. The method of claim 6, wherein the first aqueous solvent, the second aqueous solvent, or both further comprise at most 10% of a co-solvent.

8. The method of claim 7, wherein the co-solvent is DMSO, DMF, ACN, EtOH, MeOH, iPrOH, PrOH, t-BuOH, THF, DEE, DCM, acetone, or a combination thereof.

9. The method of claim 3, wherein the ¹⁸F salt is K¹⁸F, Na¹⁸F, Cs¹⁸F, ¹⁸F—K_{2.2.2}/K₂CO₃, or a crown ether ¹⁸F—NR₄, wherein R is a methyl, ethyl, propyl, butyl, or pentyl.

10. The method of claim 4, wherein the fluorinated compound is according to any one of the following formulas: 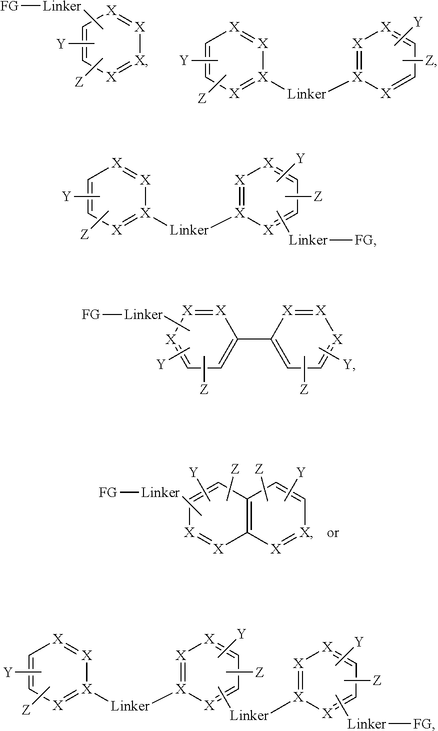
wherein X is C or N, Y is F, and Z is Cl, Br, I, NO₂, N₂, N₃, CO—NH₂, SH, SO₃H, COOH, COOR, or NR₃,
wherein R is a methyl, ethyl, propyl, butyl, pentyl, or their isomers,
wherein FG is maleimide, NHS-ester, azide, tetrazine, alkyne, or alkene,
wherein the Linker is optional or is SO₂—, SO—, CO—, CO—NH—, —O—, —S—, COO, —(CH₂CH₂O)ₙ—,
—(CO-A-NH)ₙ—, —(CO—CH(CHCH₂OH)—NH)ₙ—, —(CO—CH₂—NH)ₙ—, —(CO—CH(CH₃)—NH)ₙ—, —COC₆H₄—, —CH₂CONH—, —NCCCC₆H₄—, —NHCO—, or —NHCS—, wherein A is —(CH₂)ₙ—, and wherein n ranges from 1 to 10.

11. The method of claim 4, wherein the ¹⁸F-labeled radiotracer is used as a companion diagnostic or companion therapeutic compound for treatment or diagnostic applications.

12. The method of claim 1, wherein the target tracer compound comprises scFv, minibody, diabody, nanobody, and affibody, hormones, antibodies, glycoproteins, peptides, mRNA, siRNA, snRNA, DNA, or fragments thereof, carbohydrates, polycarbohydrates, cofactors, coenzymes, phospholipids, glycoproteins, hormones, polyethylene glycols (PEG), PEGylated biologics, PEGylated phospholipids, magnetic resonance imaging (MRI) agents, ultrasound agents, x-ray agents, computerized tomography (CT) agents, fluorescent agents, or synthetic organic or inorganic small molecules.

13. The method of claim 1, wherein providing the ¹⁸F compound comprises radiolabeling, and reacting the ¹⁸F compound with the target tracer compound comprises conjugation.

14. The method of claim 13, wherein providing the ¹⁸F compound comprises:
reacting an ¹⁸F salt with a non-radioactive fluorinated compound such that said fluorinated compound is ¹⁸F-labeled, thereby forming the ¹⁸F-compound.

15. The method of claim 14, wherein providing the ¹⁸F compound further comprises:
prior to reacting the ¹⁸F salt with the non-radioactive fluorinated compound, conjugating the non-radioactive fluorinated compound to a linker with an active functional group.

16. The method of claim 14, wherein the non-radioactive fluorinated compound has a functional group that acts as a linker for direct or indirect conjugation to the target tracer compound.

17. The method of claim 14, wherein the target tracer compound has a functional group that reacts with the ¹⁸F-labeled compound via aromatic nucleophilic substitution.

18. The method of claim 14, wherein the ¹⁸F salt is K¹⁸F, Na¹⁸F, Cs¹⁸F, ¹⁸F—K_{2.2.2}/K₂CO₃, a crown ether, or ¹⁸F—NR₄, wherein R is a methyl, ethyl, propyl, butyl, or pentyl.

19. The method of claim 14, wherein the fluorinated compound is according to any one of the following formulas: 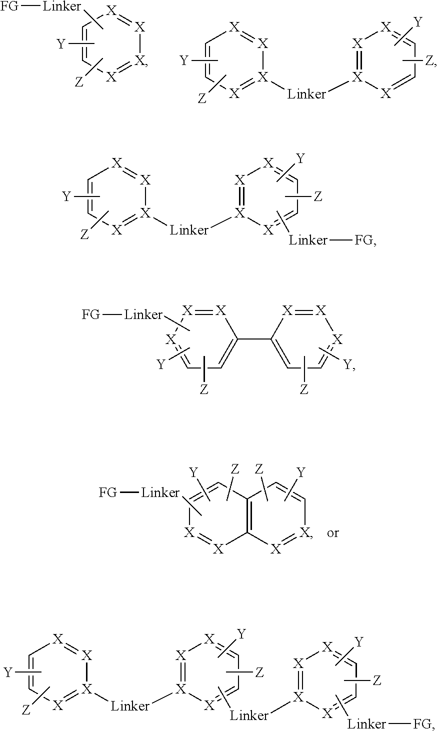
wherein X is C or N, Y is F, and Z is Cl, Br, I, NO₂, N₂, N₃, CO—NH₂, SH, SO₃H, COOH, COOR, or NR₃,
wherein R is a methyl, ethyl, propyl, butyl, pentyl, or their isomers,
wherein FG is maleimide, NHS-ester, azide, tetrazine, alkyne, or alkene,
wherein the Linker is optional or is SO₂—, SO—, CO—, CO—NH—, —O—, —S—, COO, —(CH₂CH₂O)ₙ—,
—(CO-A-NH)ₙ—, —(CO—CH(CHCH₂OH)—NH)ₙ—, —(CO—CH₂—NH)ₙ—, —(CO—CH(CH₃)—NH)ₙ—, —COC₆H₄—, —CH₂CONH—, —NCCCC₆H₄—, —NHCO—, or —NHCS—, wherein A is —(CH₂)ₙ—, and wherein n ranges from 1 to 10.

20. The method of claim 14, wherein the ¹⁸F-labeled radiotracer is used as a companion diagnostic or companion therapeutic compound for treatment or diagnostic applications.
